(19) United States
(12) Patent Application Publication
(10) Pub. No.: US 2025/0295316 A1
(43) Pub. Date: Sep. 25, 2025

(54) SYSTEMS AND METHODS FOR NON-INVASIVE IDENTIFICATION OF BIOMARKERS

(71) Applicant: ViiT Health, Inc., Mesa, AZ (US)

(72) Inventors: Luis Fernando GOMEZ, Mexico City (MX); Antonio GARCIA, Mesa, AZ (US); Jose Miguel CRUZ Y CELIS, Huixquilucan (MX); Luis Manuel Martinez MÉNDEZ, Calvillo (MX); Luis Jaime Martínez MELÉNDEZ, Mesa, AZ (US); Tiago Tito Gomes AMORIM, João Pessoa (BR); Hilarión Guadalupe Maldonado GONZÁLEZ, Manzanillo (MX); Mayra Guadalupe Mora ESPINOSA, Ciudad de México (MX); Victoria María Merino AESCHBACH, Huixquilucan (MX); Felix AGAKOV, Edinburgh (GB); Anna AGAKOVA, Edinburgh (GB)

(73) Assignee: ViiT Health, Inc., Mesa, AZ (US)

(21) Appl. No.: 19/088,717

(22) Filed: Mar. 24, 2025

Related U.S. Application Data
(60) Provisional application No. 63/568,484, filed on Mar. 22, 2024.

(51) Int. Cl. A61B 5/0205; A61B 5/00; A61B 5/026; A61B 5/053

(52) U.S. Cl. CPC A61B 5/02055; A61B 5/0002; A61B 5/7267; A61B 5/0261; A61B 5/053; A61B 2562/0238

(57) ABSTRACT

Disclosed herein are systems and methods for non-invasive identification of biomarkers. The disclosed embodiments include a portable electronic device for biosignal acquisition. The disclosed embodiments include a housing having a chamber configured to receive a sample. The disclosed embodiments include a light source array disposed adjacent to the chamber. The disclosed embodiments include a plurality of sensors configured to detect a plurality of signals from the sample. The disclosed embodiments include a tunable filter array comprising a plurality of polarizing filters. The disclosed embodiments include a communications module configured to transmit the plurality of signals.

### RELATED APPLICATIONS

This application claims priority to U.S. Provisional Application No. 63/568,464, filed Mar. 22, 2024. The disclosure of the above-referenced application is expressly incorporated herein by reference in its entirety.

### FIELD

The present disclosure relates to healthcare and wellness devices, and more particularly relates to a device that enables non-invasive identification of one or more biomarkers for disease prevention and wellness purposes, potentially contemplating diagnostic purposes, disease monitoring, and personalized healthcare.

### BACKGROUND

Identifying biomarkers such as blood glucose, oxygen saturation, heart rate, and blood pressure plays a pivotal role in comprehensive health monitoring. Blood glucose levels serve as critical indicators for diabetes prevention and management, thereby enabling timely interventions to maintain optimal wellness. Oxygen saturation measurement aids in the assessment of respiratory and circulatory health. Further, regular heart rate monitoring provides a scope into cardiovascular well-being, assisting in the early detection of abnormalities. Simultaneously, tracking blood pressure variations may help improve health by detecting risks of hypertension, heart disease, and circulatory issues.

There are various technical problems with the identification of biomarkers in conventional or traditional detection systems. Achieving high sensitivity and specificity in the determination of biomarkers is a constant challenge. Conventional or traditional technologies for the identification of biomarkers may have limitations in accuracy, sensitivity, dynamic range, and specificity according to their sensing technologies and data analysis methodologies. Further, invasive systems, such as finger pricks or implantable systems, may cause pain and/or trigger immune responses, causing decreased user inconvenience and possibly leading to the formation of scar tissue. Developing effective devices, algorithms and bioinformatics tools for data acquisition, integration, analysis, and interpretation to extract meaningful insights from complex biophysiological information is a relevant outstanding challenge.

### SUMMARY

The disclosed systems and devices may include a portable device. The disclosed embodiments may include a housing having a chamber configured to receive a sample. The disclosed embodiments may include a light source array disposed adjacent to the chamber. The light source array may be configured to emit light for transmission through the sample. The disclosed embodiments may include a plurality of sensors configured to detect a plurality of signals from the sample, the plurality of sensors including a bioimpedance sensor, a spectrometer sensor, and an infrared temperature sensor. The disclosed embodiments may include a tunable filter array including a plurality of polarizing filters. One or more polarizing filters of the plurality of polarizing filters may be oriented perpendicularly to the emitted light and disposed between the light source array and the spectrometer sensor. The disclosed embodiments may include a communications module configured to transmit the plurality of signals. The light source array, the plurality of sensors, the filter array, and the communications module may each be disposed within the housing.

In some embodiments, the light source array is configured to emit light at a plurality of wavelengths and pulsating frequencies.

In some embodiments, the plurality of polarizing filters includes at least two polarizing filters each having a different polarization state.

In some embodiments, the chamber is configured to receive the sample between the light source array and the spectrometer. In some embodiments, the one or more polarizing filters are arranged in parallel to each other and disposed between the light source array and where the chamber is configured to receive the sample.

In some embodiments, the one or more polarizing filters of the plurality of polarizing filters are arranged in parallel to each other and disposed between where the chamber is configured to receive the sample and at least one sensor of the plurality of sensors.

In some embodiments, the sample may be a peripheral anatomical sample including a vascular anatomical segment.

In some embodiments, the communications module is configured to transmit the plurality of signals to a computing system for generating a weighted ensemble biomarker determination.

The disclosed embodiments may include receiving physiological signals for a subject from a device, the physiological signals including temperature, bioimpedance, and light absorbance measurements. The disclosed embodiments may include generating a spectral footprint signal from the received physiological signals, the spectral footprint signal including Photoplethysmography (PPG) data. The disclosed embodiments may include processing the spectral footprint signal. The processing may include segmenting and generating superimposed PPG composite data. The disclosed embodiments may include extracting features from the composed PPG data to generate biomarker specific features. The disclosed embodiments may include analyzing, with one or more machine learning models, the biomarker specific features. The disclosed embodiments may include generating, based on the analysis, a weighted ensemble biomarker determination.

The disclosed embodiments may include analyzing, with the one or more machine learning models, the biomarker specific features, the physiological signals, the superimposed PPG data, and complementary data. The segmenting and generating superimposed composite data may be based on a plurality of wavelengths of the PPG data.

In some embodiments, the biomarker specific features include at least one of heart rate, heart rate variability (HRV), oxygen saturation, systolic blood pressure, diastolic blood pressure, vascular age estimation, arterial compliance, perfusion index, and respiration rate, and blood glucose.

In some embodiments, the one or more machine learning models include classical machine learning models and deep learning models, and wherein applying the one or more machine learning models comprises applying both the classical machine learning models and the deep learning model to at least one of the biomarker specific features, physiological signals, segmented PPG signals and superimposed composite PPG signals, and complementary data.

The disclosed embodiments may include a system for biomarker analysis. The disclosed embodiments may include a device including a chamber configured to receive a sample. The disclosed embodiments may include a chamber configured to receive a sample. The disclosed embodiments may include a light source array disposed adjacent to the chamber. The light source array may be configured to emit light for transmission through the sample. The disclosed embodiments may include a plurality of sensors configured to detect a plurality of signals from the sample, the plurality of sensors including a bioimpedance sensor, a spectrometer sensor, and an infrared temperature sensor. The disclosed embodiments may include a communications module configured to transmit the plurality of signals. The disclosed embodiments may include a computing system in electronic communication with the device. The disclosed embodiments may include one or more machine learning models. The disclosed embodiments may include one or more memory devices storing executable instructions and at least one processor configured to execute instructions to perform operations including receiving the plurality of signals from the device, generating biomarker specific features from the plurality of signals, and applying the one or more machine learning models to the biomarker specific features to generate a weighted ensemble biomarker determination.

In some embodiments, the operations include generating a spectral footprint signal from the received signals, the spectral footprint signal including Photoplethysmography (PPG) data and system variability data.

In some embodiments, the operations include processing the spectral footprint signal, the processing including segmenting the PPG data and generating superimposed PPG composite data, and extracting features from the composed PPG data to generate the biomarker specific features.

In some embodiments, the one or more machine learning models of the computing system includes classical machine learning models and deep learning models, and applying the one or more machine learning models includes applying both the classical machine learning models and the deep learning model to at least one of the biomarker specific features, the plurality of signals, the segmented PPG signals and superimposed composite PPG signals, and complementary data.

In some embodiments, the device includes a tunable filter array having a plurality of polarizing filters.

In some embodiments, the plurality of polarizing filters includes at least two polarizing filters each having a different polarization state.

In some embodiments, the sample includes a vascular anatomical segment.

In some embodiments, the biomarker specific features include at least one of heart rate, heart rate variability (HRV), oxygen saturation, systolic blood pressure, diastolic blood pressure, vascular age estimation, arterial compliance, perfusion index, and respiration rate, and blood glucose.

In some embodiments, the chamber, plurality of sensors, light source array, and communications module are each disposed within a housing of the device.

Embodiments including methods and computer-readable media implementing the above embodiments are also disclosed herein.

The foregoing general description and the following detailed description are example and explanatory only and are not restrictive of the claims.

### BRIEF DESCRIPTION OF THE DRAWINGS

The drawings are not necessarily to scale or exhaustive. Instead, emphasis is generally placed upon illustrating the principles of the embodiments described herein. The accompanying drawings, which are incorporated in and constitute a part of this specification, illustrate several embodiments consistent with the disclosure and, together with the description, serve to explain the principles of the disclosure. In the drawings:

FIG. 1A illustrates a block diagram of a biomarker analysis system, consistent with embodiments of the present disclosure.

FIG. 1B illustrates a view of a portable device, consistent with embodiments of the present disclosure.

FIG. 1C illustrates a view of a portable device and a sample, consistent with embodiments of the present disclosure.

FIG. 1D illustrates an exploded view of a portable device, consistent with embodiments of the present disclosure.

FIG. 1E illustrates a block diagram of a computing system, consistent with embodiments of the present disclosure.

FIG. 2A illustrates a diagram of light absorption, consistent with embodiments of the present disclosure.

FIG. 2B illustrates a graph of a spectral footprint, consistent with embodiments of the present disclosure.

FIG. 3A illustrates a diagram of Photoplethysmography, consistent with embodiments of the present disclosure.

FIG. 3B illustrates spectral footprint data, consistent with embodiments of the present disclosure.

FIG. 4A illustrates a diagram of light rotation, consistent with embodiments of the present disclosure.

FIG. 4B illustrates a diagram of polarizing filters and light rotation, consistent with embodiments of the present disclosure.

FIG. 4C illustrates spectral footprint data, consistent with embodiments of the present disclosure.

FIG. 5 illustrates a process for analyzing biomarkers, consistent with embodiments of the present disclosure.

FIG. 6 illustrates steps in the process for acquiring signals, consistent with embodiments of the present disclosure.

FIG. 7 illustrates an interface for measurement parameters, consistent with embodiments of the present disclosure.

FIG. 8 illustrates a spectral footprint, consistent with embodiments of the present disclosure.

FIG. 9 illustrates steps in the process for analyzing biomarkers, consistent with embodiments of the present disclosure.

FIG. 10 illustrates processed data, consistent with embodiments of the present disclosure.

FIG. 11 illustrates processed data, consistent with embodiments of the present disclosure.

FIG. 12 illustrates segmented data, consistent with embodiments of the present disclosure.

FIG. 13 illustrates composed data, consistent with embodiments of the present disclosure.

FIG. 14A illustrates extracted features, consistent with embodiments of the present disclosure.

FIG. 14B illustrates extracted features, consistent with embodiments of the present disclosure.

FIG. 15 illustrates a depiction of an Overall Health Index, consistent with embodiments of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

Reference will now be made in detail to exemplary embodiments, some examples of which are shown in the accompanying drawings.

It is understood that while certain embodiments are discussed to facilitate understanding of various principles and aspects of this disclosure, the embodiments are not described in isolation and the descriptions are not necessarily mutually exclusive. Thus, it is contemplated and understood that described features of principles of any embodiment may be incorporated into other embodiments.

In the present document, the words “example” or “exemplary” are used herein to mean “serving as an example, instance, or illustration.” Any embodiment or implementation of the present subject matter described herein as “exemplary” is not necessarily to be construed as preferred or advantageous over other embodiments. Any included equations are also provided as exemplary implementations.

The terms “comprise”, “comprising”, or any other variations thereof, are intended to cover a non-exclusive inclusion, such that one or more devices or sub-systems or elements or structures or components preceded by “comprises . . . a” does not, without more constraints, preclude the existence of other devices, sub-systems, additional sub-modules. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

A computer system (standalone, client or server computer system) configured by an application may constitute a “module” (or “subsystem”) that is configured and operated to perform certain operations. In one embodiment, the “module” or “subsystem” may be implemented mechanically or electronically, so a module may include dedicated circuitry or logic that is permanently configured (within a special-purpose processor) to perform certain operations. In another embodiment, a “module” or “subsystem” may also comprise programmable logic or circuitry (as encompassed within a general-purpose processor or other programmable processor) that is temporarily configured by software to perform certain operations.

Accordingly, the term “module” or “subsystem” should be understood to encompass a tangible entity, be that an entity that is physically constructed, permanently configured (hardwired), or temporarily configured (programmed) to operate in a certain manner and/or to perform certain operations described herein.

Biomarkers may include characteristics or measurements that can provide indications of health, health conditions, or changes in health. Biomarker analysis can enable an understanding of the health status of an individual. However, traditional or conventional biomarker analysis may involve invasive monitoring, and may suffer from a variety of limitations. For example, traditional glucose monitoring relies on invasive, intermittent testing (e.g., fingerstick tests, CGMs), leading to low patient adherence and limited real-time tracking. Implantable systems may trigger an immune response, leading to the formation of a scar tissue around the implantable system, which can affect the accuracy and longevity of the implantable system. Furthermore, conventional non-invasive glucose detection techniques lack accuracy due to glucose's low optical interaction compared to dominant absorbers like water and hemoglobin, and the low extinction coefficient of glucose results in a weak optical absorbance signal, making it difficult to isolate from background noise. Conventional biomarker analysis systems may suffer from poor signal-to-noise ratios and optical limitations. For example, traditional optical absorbance techniques struggle to distinguish glucose-specific signals from biological and environmental noise, tissue scattering effects further distorts light absorption and reduces accuracy, and skin tone, hydration levels, and blood perfusion variability introduce additional challenges in measurement consistency. Moreover, conventional health systems may be limited to fragmented health data, and lack personalized prevention. For example, conventional wearables or medical devices lack integration across multiple biomarkers, limiting their effectiveness in early disease detection and prevention, and provide isolated health metrics rather than a comprehensive biomarker analysis.

The disclosed embodiments address limitations in conventional or traditional biomarker analysis by providing a comprehensive and effective solution for non-invasive identification and analysis of biomarkers. The disclosed embodiments enable enhanced disease prevention, improve disease management and promote preventive healthcare strategies, fostering the overall well-being of an individual. The disclosed embodiments involve a multispectral, multisensor approach combining various sensors and analysis techniques to provide comprehensive biomarker tracking, including glucose monitoring, among other indicators. The disclosed embodiments provide enhancements in machine learning to process and analyze large amounts of acquired data to provide improved biomarker accuracy. It will be appreciated that the disclosed embodiments improve biomarker accuracy, compensate for physiological variations, enhance signal reliability, as well as strengthen biomarker estimation by correlating multiple physiological parameters.

### System Configuration

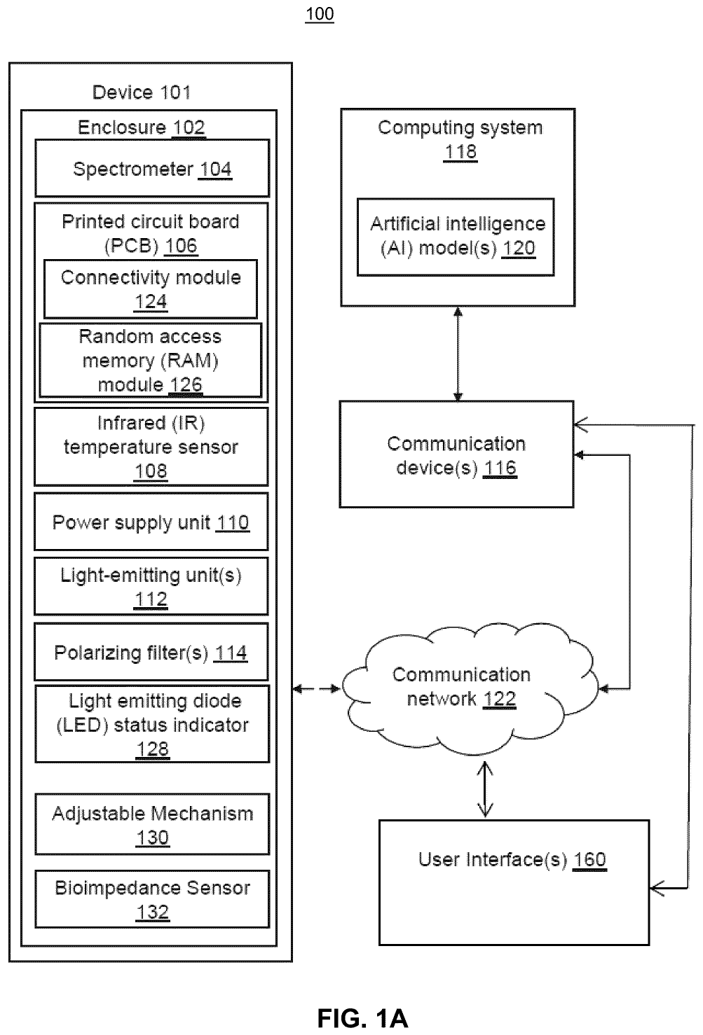

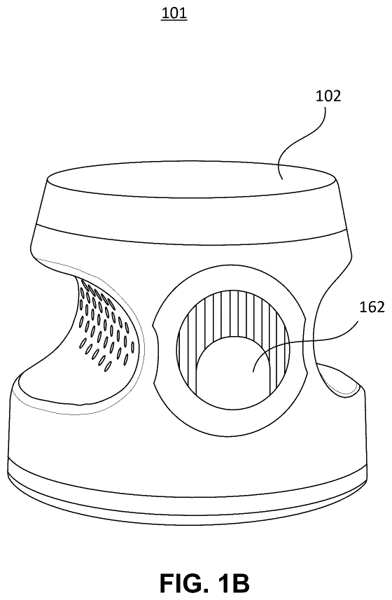

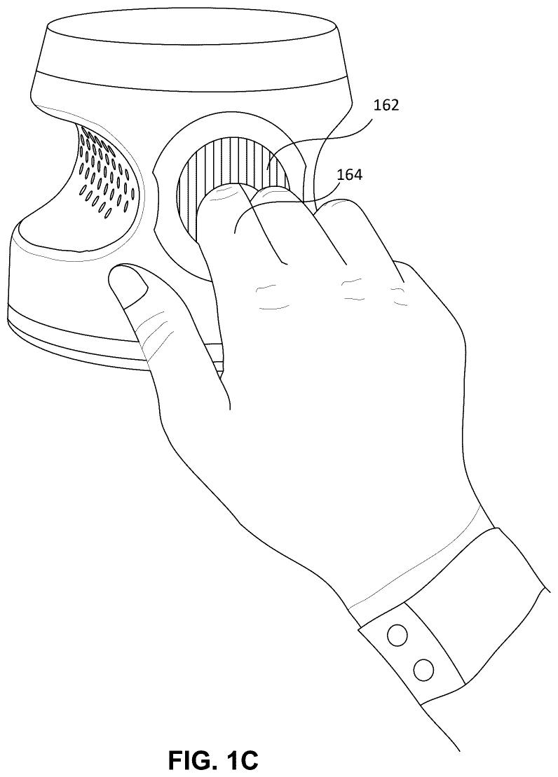

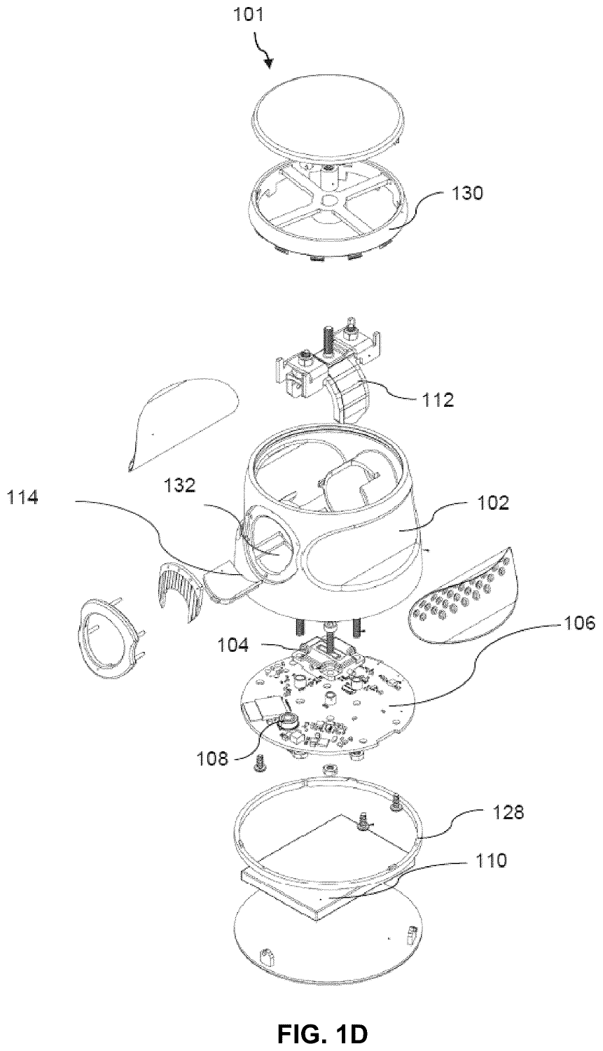

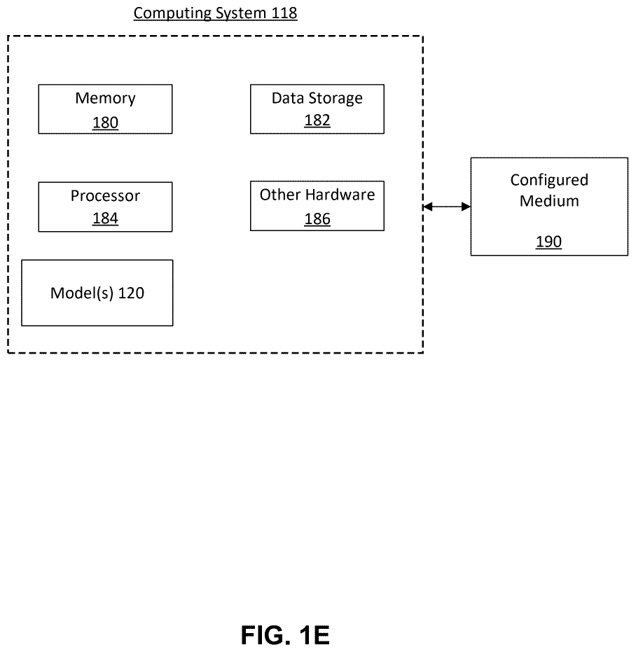

FIGS. 1A-1D illustrate a system 100 for biomarker analysis, consistent with embodiments of the present disclosure. FIG. 1A illustrates an exemplary block diagram of system 100, consistent with embodiments of the present disclosure. In some embodiments, system 100 includes a device 101. Device 101 may include a housing 102, a spectrometer 104, a Printed Circuit Board (PCB) 106, an Infrared (IR) temperature sensor 108, a power supply unit 110, a plurality of light-emitting units 112, one or more polarizing filters 114, a Light Emitting Diode (LED) status indicator 128, a connectivity module 124, a Random Access Memory (RAM) module 126, an adjustable mechanism 130, and a bioimpedance sensor 132.

In some embodiments, system 100 may include one or more of computing system 118, communication device(s) 116, and user interface(s) 160 in electronic communication with device 101. For example, computing system 118, communication device(s) 116, and/or user interface(s) 160 may be connected to device 101 via communication network 122. The communication network 122 may be a wired communication network, a wireless personal communication network, a Bluetooth low energy (BLE) a local area network (LAN), a wide area network (WAN), a metropolitan area network (MAN), a telephone network, such as the public switched telephone network (PSTN) or a cellular network, an intranet, an internet, a fiber optic network, a cloud computing network, or a combination of networks. In some embodiments, communication network 122 may transmit data to device 101 via connectivity module 124. The one or more communication devices 116 may be digital devices, computing devices, and/or networks. The one or more communication devices 116 may include a mobile device, a smartphone, a personal digital assistant (PDA), a tablet computer, a phablet computer, a wearable computing device, a laptop, a desktop, or the like. Computing system 118 may provide computing or processing capabilities for system 100. Computing system 118 may be a remote, cloud, or server computing system. User interface(s) 160 can include displays for data from system 100, such as interactive displays (e.g., capable of interacting inputs and outputs with a user). For example, user interface(s) 160 may be displayed on communication device(s) 116.

FIG. 1B illustrates a view of device 101, consistent with embodiments of the present disclosure. Device 101 may include a housing 102. Housing 102 may enclose the components of device 101. In some examples, housing 102 may be constructed from at least one of, but not limited to, a: plastic, metal, or the like. Housing 102 may include a chamber 162. Chamber 162 may be configured to receive a sample. For example, chamber 162 may have an opening that can receive a sample. In some embodiments, chamber 162 may extend throughout housing 102. Alternatively, chamber 162 may extend a portion of housing 102. Chamber 162 may form an enclosed space to enable the capture of clear signals. In some embodiments, chamber 162 may be formed as part of housing 102 (e.g., chamber 162 may be integrated with an interior portion of housing 102). In some embodiments, light emitting units 112 can form a portion of chamber 162, such as when the light array encloses a portion of chamber 162. In examples where sample 164 is a finger, housing 102 may include curvatures to sides of the device 101 to provide rest for parallel fingers. In some embodiments, device 101 includes a flexible blind at the opening of chamber 162 to block out ambient light.

FIG. 1C illustrates a view of device 101, consistent with embodiments of the present disclosure. As described herein, chamber 162 may be configured to receive sample 164. Sample 164 may be inserted into chamber 162. Sample 164 may include any sample that allows for light transmittance. Sample 164 may include a peripheral anatomic sample, such as a part of a body. For example, sample 164 may include a vascular anatomical segment (e.g., a part of the body including vasculature), such as a finger, arm, ear, foot, toe, or the like. Additionally, or alternatively, sample 164 may include a liquid-based (e.g., fluid from a subject or a solution containing fluid from a subject) or tissue-based (e.g., tissue disposed on a slide).

FIG. 1D illustrates an exploded view of device 101, consistent with embodiments of the present disclosure. PCB 106 may embed various modules and/or sensors of device 101. For example, the connectivity module 124 and the RAM module 126 of the device 101 may be embedded in the PCB. In some embodiments, PCB 106 may include a processor or microprocessor. The PCB 106 may include a microcontroller unit (MCU) to function as a central control unit responsible for overseeing the control of the device 101, communication function of device 101, the connectivity module 124, and the RAM module 126. The MCU may include a processor or microprocessor. In some embodiments, PCB 106 may also include storage. The connectivity module 124 is configured to connect with a communication network 122. The RAM module 126 within the device 101 may facilitate the installation of firmware updates. This RAM module 126 configuration enables the smooth incorporation of new software enhancements, bug fixes, and feature updates, thereby improving the overall functionality and performance of the device 101.

In some embodiments, device 101 can include a light source or a plurality of light sources. Device 101 may include a light source array, which may be configured as a plurality of light-emitting units 112. The plurality of light-emitting units 112 may be arranged to radiate the light from several angles towards a human finger, or other body parts. For example, the plurality of light-emitting units 112 may be arranged in the form of a half circle. The plurality of light-emitting units 112 can support multiple wavelength ranges at the same time and may be programmed to allow for the generation of different pulsating light patterns. Light-emitting units 112 may include a plurality of Light-Emitting Components, including but limited to Light-Emitting Diodes (LEDs) or Lasers that emit a light in the range of 300 nanometers to 3000 nanometers. For example, the plurality of light-emitting units 112 can support three wavelength ranges, such as ranges in visible (e.g., 660 nm), Near-infrared (e.g., 870 nm), and Mid-infrared (MID-IR) (e.g., 3000 nm). In some embodiments, the plurality of light-emitting units 112 can be controlled (e.g., by PCB 106) to modulate which light units are turned on or off, the wavelength of lights, as well as the duration of the light unit being turned on or off, thereby providing a controllable light source capable of emitting light of different wavelengths and from different angles. The plurality of light-emitting units 112 may also include a controller and connector for effective operation of the device 101. The adjustable mechanism 130 can be used to adjust the distance of the plurality of light-emitting units 112 to the analyzed sample. The adjustable mechanism 130 may displace the plurality of light-emitting units 112 perpendicular to the sample, according to different sample sizes (e.g., thereby accommodating different finger sizes and reducing ambient signal noise).

In some embodiments, device 101 may include a filter array formed by one or more polarizing filters 114. The one or more polarizing filters 114 can filter the light oscillating in specific directions, aligned at angles ranging between 0° and 180° to the direction of propagation of the light (e.g., from light-emitting units 112). The one or more polarizing filters can involve various polarization states, which can include the angles of the polarizing filter (e.g., thereby filtering out light at such angles). Exemplary angles can include 0°, 45°, and 90° with respect to light-emitting units 112. The one or more polarizing filters 114 can be disposed between light-emitting units 112 and the sample to provide pre-sample analysis (e.g., before light reaches the sample). Additionally, or alternatively, the one or more polarizing filters 114 can be disposed between the sample and a sensor to provide post-sample analysis (e.g., after light interacts with the sample). In some embodiments, the one or more polarizing filters 114 may be disposed parallel to each other. In some embodiments, one or more polarizing filters of filters 114 may be oriented perpendicular to the light-emitting units 112 and/or perpendicular to light emitted from light-emitting units 112. The one or more polarizing filters 114 may include Liquid Crystal Displays (LCDs) and/or analog filters positioned at different positions. The filter array may be tunable. The alignment of the angles, as well as activation of the filters, may be dynamically changed from 0° to 180°. For example, based on instructions received by device 101, device 101 may move individual filters of the one or more polarizing filters 114 to different angles or to different positions with respect to the light-emitting units 112, sample 164, and spectrometer 104 (e.g., filters can be moved between such components or moved away from such components to provide adjustable filtering).

In some embodiments, device 101 may include a plurality of sensors, such as spectrometer 104, IR sensor 108, and bioimpedance sensor 132. Spectrometer 104 may have a sensibility that ranges from 300 nanometers to 3000 nanometers. For example, spectrometer 104 may include a visible to MIR (mid infrared range). Spectrometer 104 may assist in capturing Vis to MIR spectral data to enhance glucose detection and assess vascular health. The Vis to MIR spectral data can provide complementary optical information to improve signal interpretation and biomarker accuracy. In some examples, spectrometer 104 can provide a voltage based on measured light. IR temperature sensor 108 may assist in tracking body temperature variations, as well as compensating for temperature-dependent metabolic changes that can influence glucose levels. IR temperature sensor 108 may complement the one or more biomarkers measurements of the device 101. The IR temperature sensor 108 can detect the radiation emitted by the human body and convert detected wavelength(s) to temperature by employing Wien's Displacement Law. IR sensor 108 can assist in adjusting calibration models based on thermal fluctuations that can affect optical properties. IR temperature sensor 108 may be configured for a lower energy range as compared to the spectrometer 104. IR temperature sensor 108 may include an emittance spectroscopy thermometer, IR pyrometer, IR thermocouple, or the like, as non-limiting examples. Bioimpedance sensor 132 may include any sensor or instrument that can measure the resistance to electrical current of the sample. Bioimpedance sensor 132 can measure hydration levels of the sample, which can correlate with glucose fluctuations and impact optical signal consistency. Bioimpedance sensor 132 can help adjust glucose estimations by compensating for fluid shifts that affect absorption properties. In some embodiments, device 101 may include a plurality of each of spectrometer 104, IR sensor 108, and/or bioimpedance sensor 132. In an exemplary configuration, the resolution of the spectrometer 104 may be 3 to 18 nm, the light path of the device 101 may be adjusted from 1.5 cm to 2.5 cm using the adjustable mechanism 130, and the pulsating light frequency of the device 101 may be adjusted from 1 Hz to 150 Hz. It will be appreciated that the configuration of sensors in device 101 can improve biomarker accuracy, compensate for physiological variations, and enhance signal reliability, as well as strengthen biomarker estimation by correlating multiple physiological parameters, and reduce errors caused by environmental or biological factors.

Device 101 may include various feedback mechanisms. For example, device 101 can include status indicator 128. Status indicator 128 can emit a light or sound, such as emitting different colors or sounds depending on the status of device 101. Status indicator 108 may be a Light Emitting Diodes (LEDs) embedded in the PCB 106 that circle the base and provide visual cues to signify a status of the device 101. The status may include whether the device 101 is powered on, calibrating, actively conducting a measurement, syncing data via the communication network 122, undergoing a server/cloud computing system 118 update, and the like. Status indicator 128 may also provide feedback on whether the sample is positioned correctly or incorrectly within device 101. Status indicator 128 can include speakers to emit audio feedback.

In some embodiments, device 101 may be a portable and rechargeable electronic device that measures the one or more biomarkers via light projected to sample 164. Device 101 may include a power supply unit 110. For example, power supply unit 110 can provide up to 30 hours of continuous operation on a single charge to the device 101. Additionally, or alternatively, device 101 may include wired connections to an external power source or be configured to receive a wired connection.

FIG. 1E illustrates a block diagram of computing system 118, consistent with embodiments of the present disclosure. As described herein, computing system 118 may be implemented via hardware, over server, over the cloud, or the like. Components of computing system 118 may include, but are not limited to, various hardware components, such as one or more processors 184, data storage 182, a system memory 180, other hardware 186, and a system bus (not shown) that couples (e.g., communicably couples, physically couples, and/or electrically couples) various system components such that the components may transmit data to and from one another. The computing system 118 may include a uniprocessor or multiprocessor computing device, or more computing devices (e.g., multiple computing devices 502) in a given computer system, which may be clustered, part of a local area network (LAN), part of a wide area network (WAN), client-server networked, peer-to-peer networked within a cloud, or otherwise communicably linked. A computer system may include an individual machine or a group of cooperating machines. A given computing system 118 may be configured for end-users, e.g., with applications, for administrators, as a server, as a distributed processing node, as a special-purpose processing device, or otherwise configured to train machine learning models and/or use machine learning models.

Computing system 118 includes at least one logical processor 184. The at least one logical processor 184 may include circuitry and transistors configured to execute instructions from memory (e.g., memory 180). For example, the at least one logical processor 184 may include one or more central processing units (CPUs), arithmetic logic units (ALUs), Floating Point Units (FPUs), and/or Graphics Processing Units (GPUs). The computing system 118, like other suitable devices, also includes one or more computer-readable storage media, which may include, but are not limited to, memory 180 and data storage 182. In some embodiments, memory 180 and data storage 182 may be part of a single memory component. The one or more computer-readable storage media may be of different physical types. The media may be volatile memory, non-volatile memory, fixed in place media, removable media, magnetic media, optical media, solid-state media, and/or of other types of physical durable storage media (as opposed to merely a propagated signal). In particular, a configured medium 190 such as a portable (i.e., external) hard drive, compact disc (CD), Digital Versatile Disc (DVD), memory stick, or other removable non-volatile memory medium may become functionally a technological part of the computer system when inserted or otherwise installed with respect to one or more computing systems 118, making its content accessible for interaction with and use by processor(s) 184. The removable configured medium 190 is an example of a computer-readable storage medium. Some other examples of computer-readable storage media include built-in random access memory (RAM), read-only memory (ROM), hard disks, and other memory storage devices which are not readily removable by users (e.g., memory 180). In some embodiments, configured medium 190 may be non-transitory. The configured medium 190 may be configured with instructions (e.g., binary instructions) that are executable by a processor 184; “executable” is used in a broad sense herein to include machine code, interpretable code, bytecode, compiled code, and/or any other code that is configured to run on a machine, including a physical machine or a virtualized computing instance (e.g., a virtual machine or a container). The configured medium 190 may also be configured with data which is created by, modified by, referenced by, and/or otherwise used for technical effect by execution of the instructions. The instructions and the data may configure the memory or other storage medium in which they reside; such that when that memory or other computer-readable storage medium is a functional part of a given computing device, the instructions and data may also configure that computing device.

In some embodiments, data storage 182 can store data received from device 101. Data storage 182 can also include or access training data for AI model(s) 120. For example, data storage 182 can include training datasets that include electrochemical-spectrophotometric venous blood glucose tests (gold standard), lab performed oxygen saturation tests, sphygmomanometer blood pressure measurements, bioimpedance body composition analysis, blood chemistry analysis, or other clinically relevant data points. Training allows the models to establish strong correlations for both primary and secondary predictive objectives (e.g., health conditions and specific health complications due to underlying conditions respectively). AI model(s) 120 can include a plurality of machine learning models, such as Lasso Regression, Support Vector Regression, Gaussian Process Regression for biomarker estimation and trend analysis, as well as deep learning models (e.g., Recurrent Neural Networks, Transformers, multi-task models), or the like. In some embodiments, computing system 118 may be connectable to the internet, and may access datasets accessible via the internet, such as training datasets or datasets having data specific to various cohorts (e.g., diabetics, athletes, elderly individuals). In some embodiments, storage 182 can store presets for device 101, such as data that may be specific to a given device, given user, or cohort similar to the user. For example, storage 182 may store previous measurements for a user, and computing system 118 may access the stored measurements when a user inputs a User ID into an interface of interface(s) 160.

### Approach

In some embodiments, device 101 may employ a transmittance absorbance spectroscopy (TAS), Photoplethysmography (PPG), and light rotation (LR) to identify the presence of one or more biomarkers and/or physiological variables non-invasively. The biomarkers and physiological variables may include glucose, oxygen saturation, heart rate, blood pressure, respiratory rate, perfusion index, temperature, vascular aging, bioimpedance, body water, cholesterol, triglycerides, oxidative stress or the like.

The device 101 can implement a multi-wavelength spectroscopic technique by employing alternating the Visible and Infrared light within a wavelength range that may be from 300 nanometers to 3000 nanometers, but not limited to it, and may include wavelength ranges that may exceed 3000 nanometers. The light may be emitted at an adjustable pulsating frequency of 1 hertz (Hz)-150 Hz, to identify volumetric variations through absorbance and rotation in a sample throughout a specific measurement time. The pulsated light may transmitted through the sample (e.g., a finger and/or other peripheral anatomic structures), within the chamber 162, to create clear spectral footprints In some examples, the spectral footprints can be taken within a 30-second individual measurement time frame, but could be shorter or longer

Embodiments of the present disclosure may involve spectroscopy. Spectroscopy includes a light source, the sample to be analyzed, and a light detector and sensor. The light directed to the analyzed sample engages with the sample constituent substances, leading to modifications in the “incident light” through processes like absorption, reflection, refraction, and transmission, producing a “resulting light.” The interpretation of disparities between “incident light” and “resulting light” enables the extraction of meaningful information about the analyzed sample.

Spectroscopy can be categorized according to observed optical phenomena and particular energy ranges of the electromagnetic radiation. The energy of a photon is a function of its frequency and wavelength, in such a way that shorter wavelengths have higher energy while larger wavelengths have lower energy. The electromagnetic energy interacts with the samples in several ways by inducing changes in the vibrational states of molecules, and by generating electronic transitions in atoms that form the analyzed molecules.

In some embodiments, device 101 employs NIR light, as the wavelength range may penetrate a human tissue better than the visible light without damaging it. Furthermore, the NIR light exhibits weaker absorption bands from water than middle IR and far IR, thereby minimizing information loss when analyzing liquid samples while also reducing heating. The NIR light holds several sensible ranges within its broadband that generate vibrational molecular absorbance of important biomolecules that relate to the several physiological variables. Additionally, the NIR light may be employed with accessible infrastructure and may provide arrangement flexibility, making the NIR ideal for identifying various biochemical analytes associated with the one or more biomarkers in a human body. NIR spectroscopy may be advantageous as it can be non-destructive (compared to UV) and produces low heat (compared to IR/microwaves). NIR interacts with overtone and combination bands of molecular vibrations, particularly in C—H, O—H, and N—H bonds, which are key components of biomolecules like glucose, proteins, and lipids. This enables highly specific biochemical identification. Further, NIR can be optimized for specific functional groups in biomolecules, with multi-wavelength approaches (e.g., 660, 870, and 940 nm), enhancing selectivity and accuracy in identifying physiological markers.

Spectroscopy can include emittance spectroscopy, transmittance spectroscopy, and reflectance spectroscopy. Both transmittance and the reflectance spectroscopy may be focused on analyzing absorbance, refraction, dispersion, and scattering to different extents. In some embodiments, device 101 provides transmittance-absorbance spectroscopy in the Vis to MIR wavelength ranges given that such technique provides the possibility to analyze a larger area than reflectance spectroscopy and may provide the full spectral footprint of the analyzed sample because of its penetration.

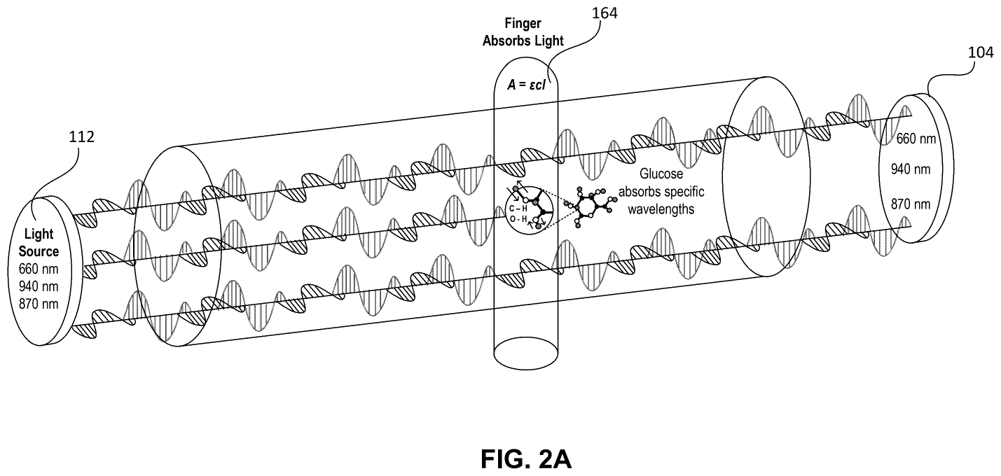

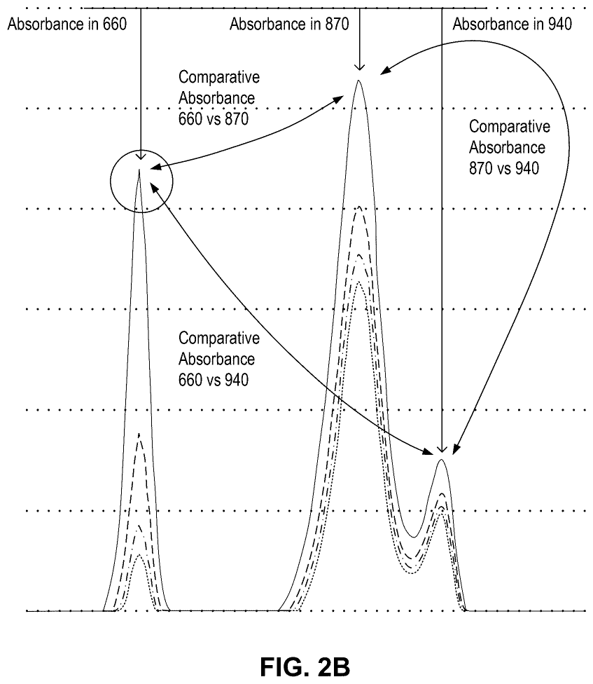

FIG. 2A illustrates a sketch of Transmittance-Absorbance Spectroscopy (TAS), consistent with embodiments of the present disclosure. In some embodiments, the TAS range can include wavelengths of 300 to 3000 nm. Light-emitting units 112 may emit light at varying wavelengths, such as 660 nm, 940 nm, and 870 nm, which can provide a broader sample footprint and enhance measurement accuracy. The light may be transmitted through sample 164. Spectrometer 104 may measure light transmittance through sample 164 (e.g., by comparing incident vs. resulting light intensity to assess spectral signals). For example, glucose in sample 164 may absorb specific wavelengths, and device 101 may identify the amount of light that gets absorbed for each wavelength. The disclosed embodiments involving TAS may provide a steadier signal given its proclivity to be less affected by the ambient light. TAS can simultaneously analyze oxygenation levels, hemoglobin concentration, hydration status, and metabolic changes, making it a versatile tool for preventive healthcare. Moreover, TAS may involve reduced sample preparation effort (e.g., as compared to conventional biochemical assays), making it more practical for portable and point-of-care diagnostics.

FIG. 2B illustrates a spectral footprint including specific absorbances, consistent with embodiments of the present disclosure. In some embodiments, a wavelength of 660 nm may provide strong interaction with hemoglobin, useful for oxygen saturation and vascular assessments; a wavelength of 870 nm may provide effective measuring of tissue perfusion and other physiological parameters; a wavelength of 940 nm may provide sensitivity to glucose absorption, making it optimal for non-invasive glucose monitoring. It will be appreciated that a combination of such wavelengths may provide enhanced biomarker detection, enabling system 100 to simultaneously assess multiple biomarkers, thereby improving accuracy and reliability in detecting glucose levels and other vital physiological indicators.

In some embodiments, the disclosed embodiments may involve Photoplethysmography (PPG). PPG is an optical technique that studies the variation of light intensity when interacting with a particular substance through time. PPG can be employed to identify the volumetric changes in microvascular tissue by recording the light transmittance, reflection, and scattering, and further analyzing the waveform signal created by the resulting light. The resulting light may be comprised of a pulsatile alternating current (AC) component attributed to cardiac synchronous changes in a blood volume with each heartbeat, and a non-pulsatile direct current (DC) component attributed to respiration, sympathetic nervous system activity, thermoregulation, or the like.

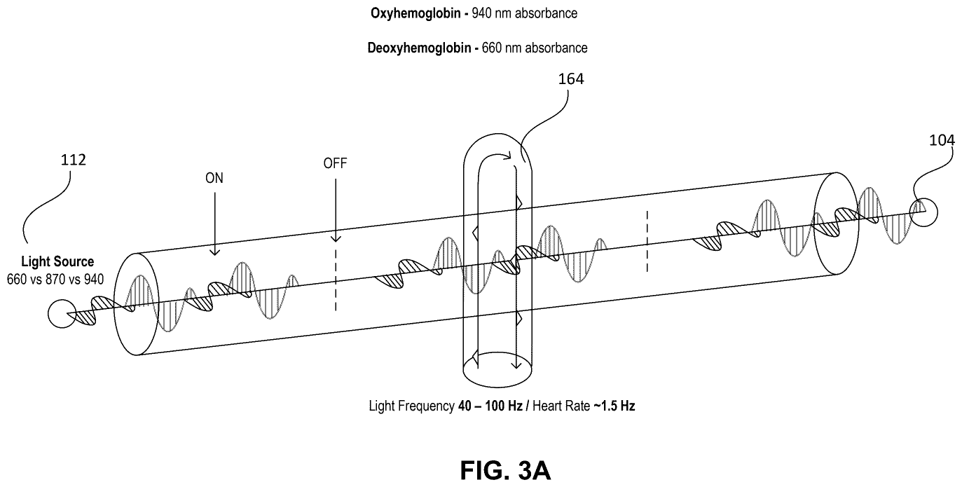

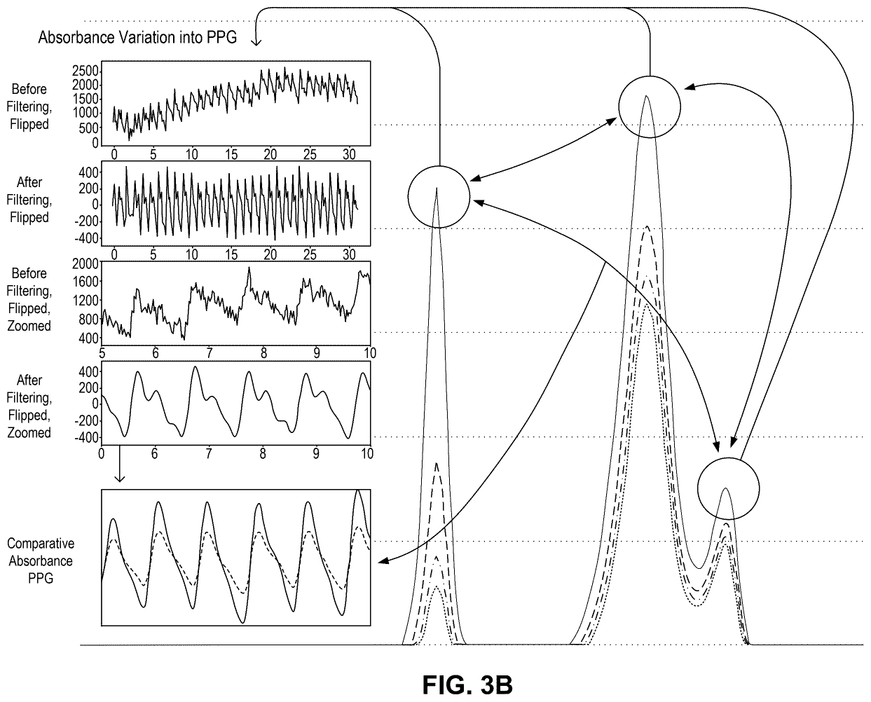

FIG. 3A illustrates a diagram of PPG implementation, consistent with embodiments of the present disclosure. Device 101 may employ PPG to generate Near Infrared Transmittance Absorbance (TAS) spectroscopic signals using frequency ranges from 40 Hz to 150 Hz. For example, device 101 may pulsate wavelengths from light-emitting units 112 at red (e.g., 660 nm) and infrared (e.g., 940 nm), and comparing the absorbance in sample 164, while using 870 nm as a reference. Such a configuration may enable the identification of deoxyhemoglobin and oxyhemoglobin when comparing the pulsated light absorbance of wavelengths in the red range and the infrared range. The oxyhemoglobin absorbs more infrared light, and the deoxyhemoglobin absorbs more red light.

FIG. 3B illustrates a sketch of PPG applied to a spectral footprint, consistent with embodiments of the present disclosure. By analyzing variations in the TAS spectroscopic signals, the device 101 can acquire insights related to cardiovascular health, sympathetic nervous health, blood oxygenation, heart rate, and other insights regarding other biomarkers. It will be appreciated that PPG may allow estimation of blood pressure without a cuff by combining AI and/or Pulse Transit Time (PTT) analysis. PPG may be used to estimate Blood Glucose concentrations by analyzing sympathetic nervous system responses to glucose variations. HRV, derived from PPG signals, provides insights into autonomic nervous system activity, which is influenced by glucose metabolism. PPG may be used in aging and vascular health analysis, since PPG-derived metrics like arterial compliance, pulse wave analysis, and endothelial function indicate vascular condition and aging-related change. PPG can be used to detect breathing patterns and assess cardiorespiratory fitness and recovery in athletes. PPG can assist in early detection of health issues, given that changes in PPG signals can indicate circulatory problems, dehydration, autonomic dysfunction, or early signs of conditions like hypertension, diabetes, or arrhythmias.

In some embodiments, the disclosed embodiments may involve light rotation (LR). It will be recognized that when light interacts with anisotropic materials—which have direction-dependent physical properties—it experiences a phase shift due to variations in the refractive index along different molecular orientations. This occurs because the refractive index depends on the alignment of the material's structure relative to the oscillation direction of the light wave. As a result, the light's polarization state can change (this is the basis for elliptical or circular polarization). (LR) can be used to detect biomarkers, including to detect glucose concentration, leveraging its particular optical property known as chirality. Chiral molecules, such as glucose, lack mirror symmetry (e.g., having a non-superposable mirror image) and can rotate the plane of polarized light, with the rotation angle proportional to their concentration. This occurs because, as light interacts with sequential chiral molecules, it undergoes a phase shift due to their anisotropic nature. Unlike symmetric molecules (e.g., water), which cancel out such shifts, chiral molecules accumulate the rotation effect, leading to measurable light rotation after multiple interactions. This property enables the quantification of glucose concentration based on its optical activity.

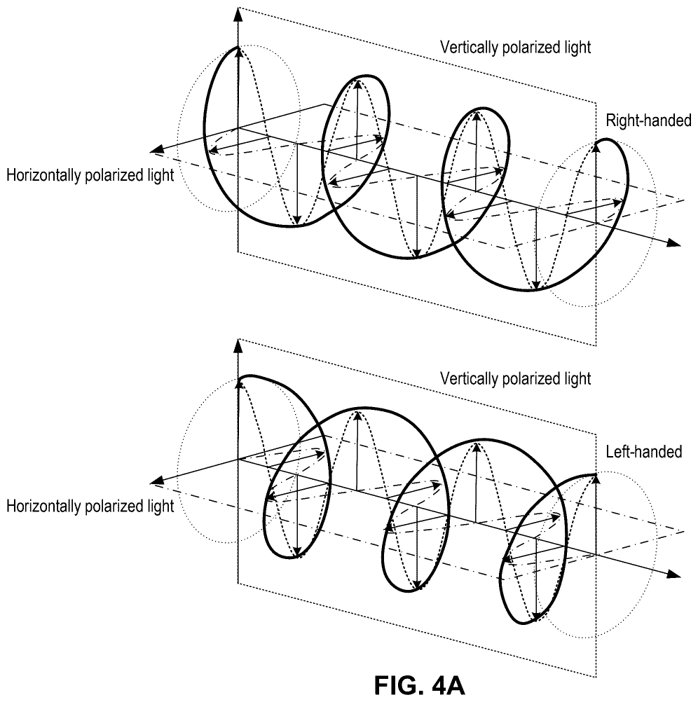

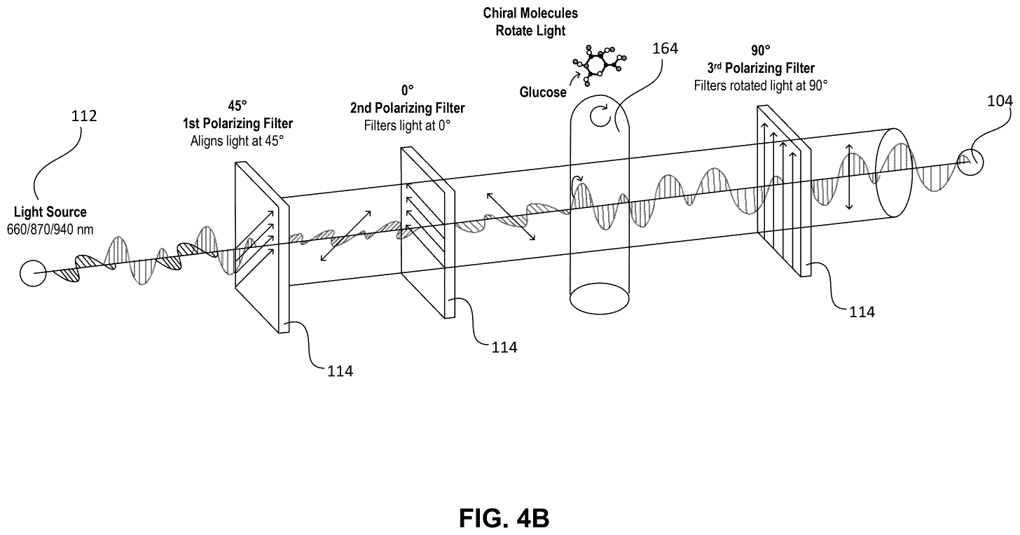

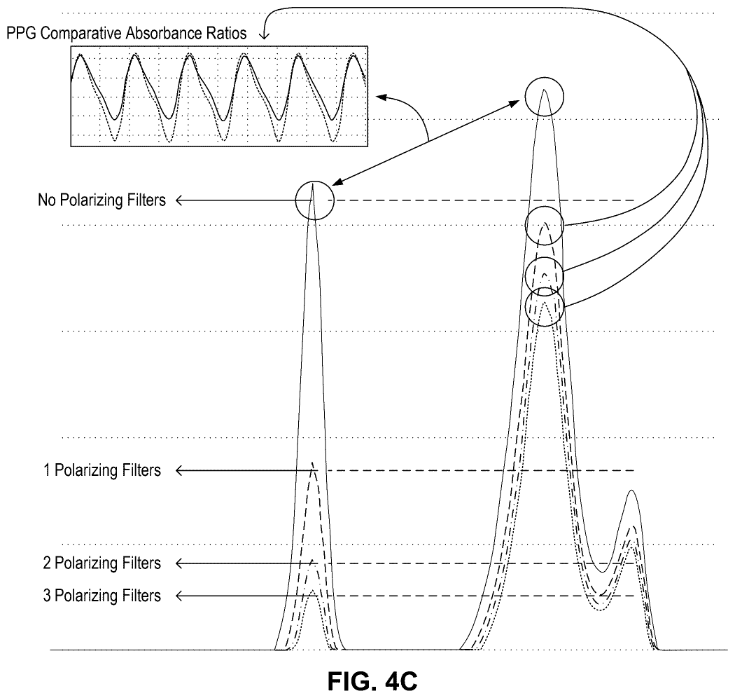

FIG. 4A illustrates light rotation, consistent with embodiments of the present disclosure. In some embodiments, chiral molecules (e.g., glucose) can rotate plane-polarized light in opposite directions, as illustrated in FIG. 4A.

FIG. 4B illustrates light rotation applied to a sample, consistent with embodiments of the present disclosure. Light emitting units 112 may emit light at various wavelengths as described herein, such as 660, 870, and 940 nm. In some embodiments, one or more polarizing filters 114 may be disposed between light-emitting units 112 and the spectrometer 104. Polarizing filter arrays 114 may manipulate the direction of light wave oscillation, thereby allowing a certain amount of light to pass through the sample 164 in different quantities and reduce scattering. The spectrometer 104 may make intensity measurements of the transmitted light with different filter configurations, and subsequently generate comparisons between them while taking into account relevant sample properties. For example, a first polarization filter may be disposed between light-emitting units 112 and the sample 164. The first polarization filter may have an angle of 45 degrees with respect to the light, thereby aligning the light at 45 degrees. A second polarization filter may be disposed between the first polarization filter and sample 164. The second polarization filter may have an angle of 0 degrees, such that it can filter light at 0 degrees. A third polarization filter may be disposed between sample 164 and spectrometer 104. The third polarization filter may have an angle of 90 degrees, such that it can filter light at 90 degrees. It will be appreciated that filtering can enhance glucose detection sensitivity by minimizing interference from non-glucose absorbers and scattering effects.

FIG. 4C illustrates LR applied to a spectral footprint, consistent with embodiments of the present disclosure. It will be appreciated that a LR configuration as described herein can amplify direct glucose information. In some embodiments, the LR technique can be further coupled with TAS absorbance and PPG data to provide a more comprehensive glucose concentration assessment.

### Implementation

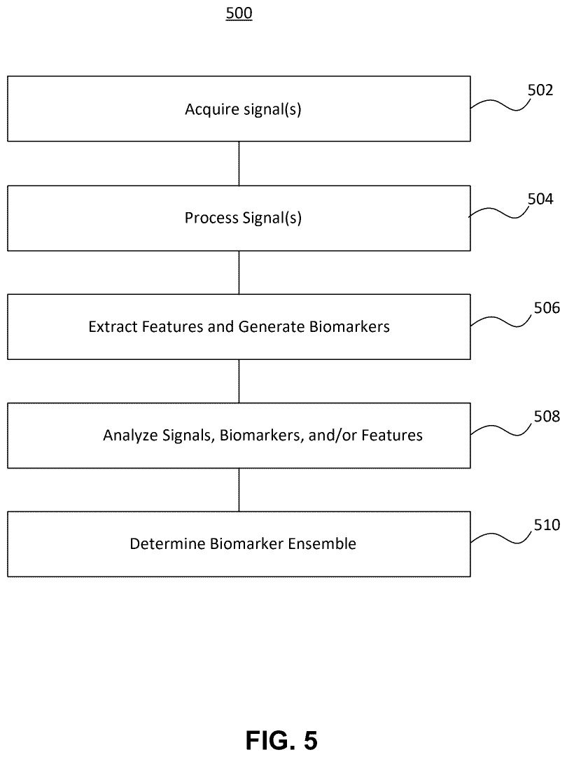

FIG. 5 illustrates a flow diagram of a process 500 for analyzing biomarkers, consistent with embodiments of the present disclosure. In some embodiments, system 100 may perform one or more steps in process 500. In some embodiments, process 500 includes a step 502 of signal acquisition, a step 504 of signal processing, a step 506 of feature extraction and biomarker generation, a step 508 of biomarker, feature and/or signal analysis, and a step 510 of biomarker determination.

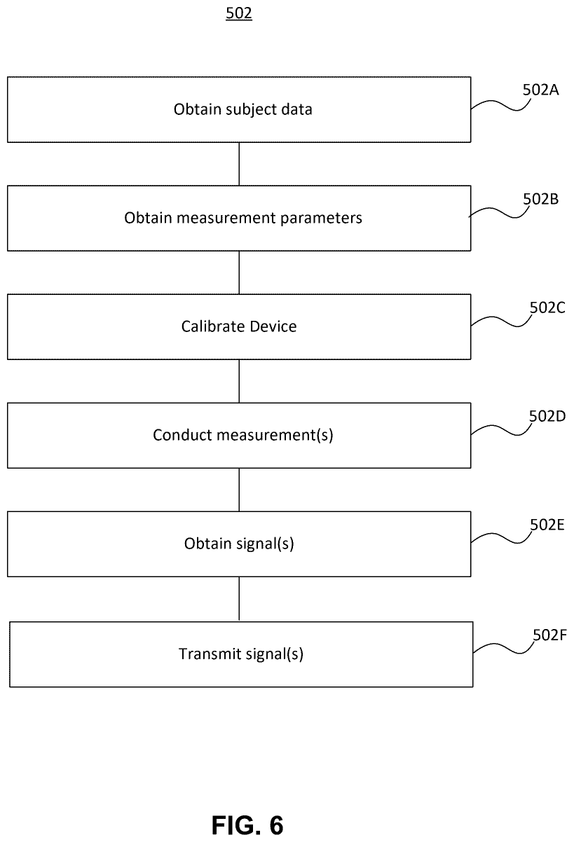

Process 500 may include a step 502 of signal acquisition. Signal acquisition may involve obtaining information, data, or measurements for a subject. Signal acquisition may occur over a plurality of scans. In some embodiments, step 502 may involve one or more substeps. FIG. 6 illustrates a flow diagram of substeps of step 502, consistent with embodiments of the present disclosure. Substep 502A may involve obtaining data that may be complementary to measured signals. For example, complementary data may include subject data, such as demographic data, anthropometric data, physiological data, health history, or the like. For example, subject data may include sex, age, weight, height, family history, pre-existing conditions, previous medical history, skin tone, or the like. Subject data can be received from user interface(s) 160 (e.g., inputted by the subject or an operator of system 100), or obtained from a database (e.g., storage 182 may contain subject data). Complementary data may also include environmental data, such as location, time of measurement, time since last meal, or the like.

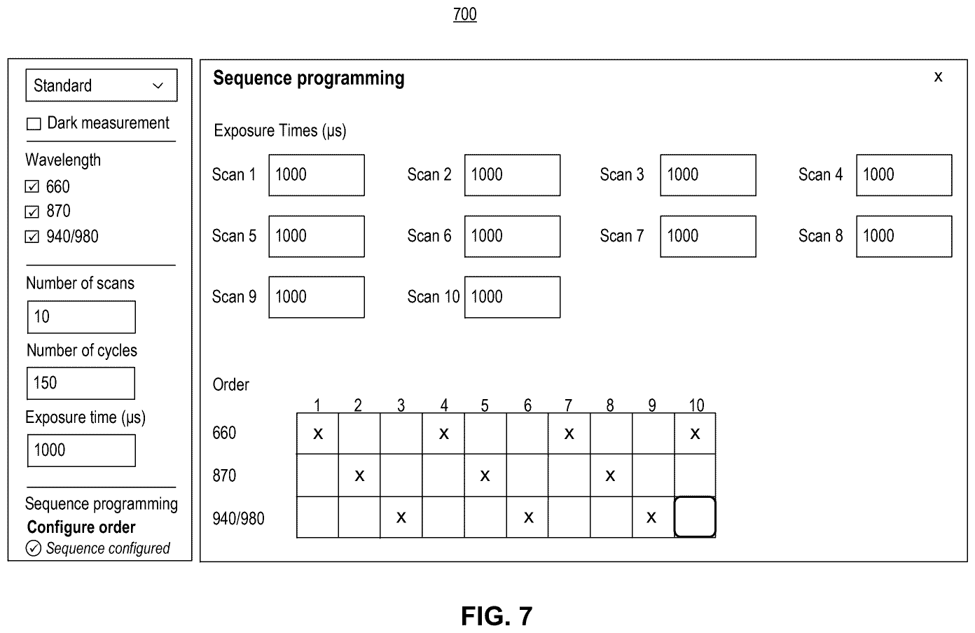

Substep 502B may involve obtaining measurement parameters for device 101, such as tunable parameters, sensor parameters, and measurement characteristics. FIG. 7 illustrates an exemplary measurement selection interface 700 for obtaining measurement parameters, consistent with embodiments of the present disclosure. As an example, user interface(s) 160 may include measurement selection interface 700. Tunable parameters can include measurement pattern, number of scans, number of cycles, selected wavelengths (300-3000 nm [visible-MIR]), light source pattern (e.g., number and order of active Light Emitting Components of the light-emitting units 112), sensor integration time per scan, and selected polarizing filters. For example, tunable parameters may be obtained from user interface(s) 160 or stored in storage 182 as preset configurations. Interface 700 may include measurement characteristics, such as selections for a mode of measurement, dark measurement (ambient light), time of measurement, sample size (ergonomic volumetric sample measurement), light path (distance measurement from light source to sensor), device calibration coefficients, location of measurement, or the like.

For example, for a pattern, substep 502B may include selecting active wavelengths, number of scans, light source order, one or more polarizing filters 114 to be active, scans per polarizing filter reading, order of polarizing filters, and the integration time per scan in the pattern, and number of cycles. It will be appreciated that combinations of different wavelengths of light can be selected during different scans to enhance the acquired signal. Additionally, for continuous measurement (e.g. monitor mode), substep 502B may include selecting the active wavelengths, the active polarizing filters, and the integration time.

Referring again to FIG. 6, step 502 may include a substep 502C of calibrating device 101, as will be described herein, and a substep 502D. Substep 502D may include conducting measurement(s). In substep 502D, the sample 160 can be placed in the chamber 162. Based on the tunable parameters, device 101 may pulsate wavelengths of light from the light-emitting units 112, and device 101 may scan sample 164 to obtain a spectral footprint. Different light-emitting units 112 may be turned on for differing amounts of time, based on the measurement parameters. The pulses of light-emitting units 112 may match the photonic capturing frequency defined by the integration time of spectrometer sensor 104. In addition, the measurement pattern may activate one or more polarization filters 114. Step 502D may involve one or more scans, and the scans may involve TAS and LR, as described herein. For example, the transmitted light may experience absorbance as it interacts with sample 164, and the one or more polarizing filters 114 may filter light with specific alignment and detect rotation effects as the light reaches spectrometer 104. In some embodiments, a first polarization filter may be disposed between light-emitting units 112 and the sample 164 to filter light at 45 degrees from the direction of light propagation, a second polarization filter may be disposed between the first polarization filter and sample 164 to filter light at 0 degrees from the direction of light propagation and parallel to the previous polarizing filter, and a third polarization filter may be disposed between sample 164 and spectrometer 104 to filter light at 90 degrees from the direction of light propagation and parallel to the previous polarizing filter. Substep 502D may also involve obtaining measurements from bioimpedance sensor 132 and IR sensor 108.

In an example, measurements can be collected at 23 millisecond time steps, resulting in a 40-50 Hz frequency, utilizing spectral data from 600 to 1000 nm, with specific wavelengths at 660, 870, and 940 nm. For a regular measurements, the pulsating light is emitted by Light-emitting unit(s) 112, then it passes through the one or more polarizing filters 114 if selected, then through the sample 164, then through the one or more polarizing filters 114 if selected, and then to the spectrometer sensor 104.

Step 502 may include a substep 502E of obtaining one or more signals from device 101. In some embodiments, a plurality of signals obtained can include the spectral footprint data from spectrometer 104, as well as complementary data from bioimpedance sensor 132, the IR sensor 108 and other metadata. For example, the obtained signals can include a spectral footprint composed of scans, an bioimpedance measurement, and a temperature measurement, from respective sensors 104, 132, and 104.

Step 502 may involve a substep 502F of transmitting the one or more signals. For example, connectivity module 124 may transmit the signals from device 101 to computing system 118 over communication network 122. In some embodiments, step 502F may involve computing system 118 obtaining the plurality of signals. For example, computing system 118 may receive the signals over communication network 122. The signals may be transmitted as one or more comma separated value (CSV) files, voltage or current values, JSON, XML, spreadsheet files, image files (e.g., TIFF, PNG, JPEG), or the like, as non-limiting examples.

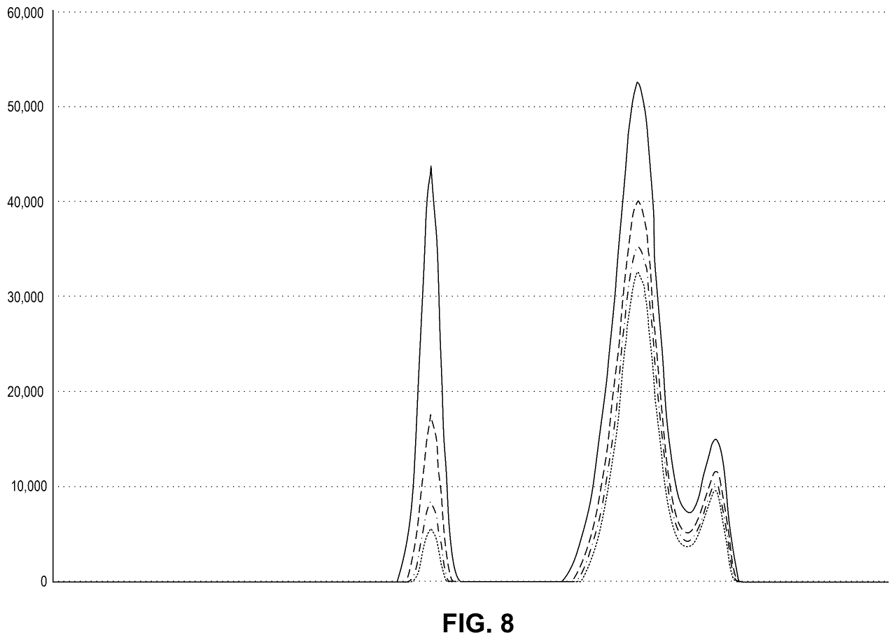

FIG. 8 illustrates a graph representation of a resulting spectral footprint, consistent with embodiments of the present disclosure. It will be appreciated that the spectral footprint includes different wavelength peaks.

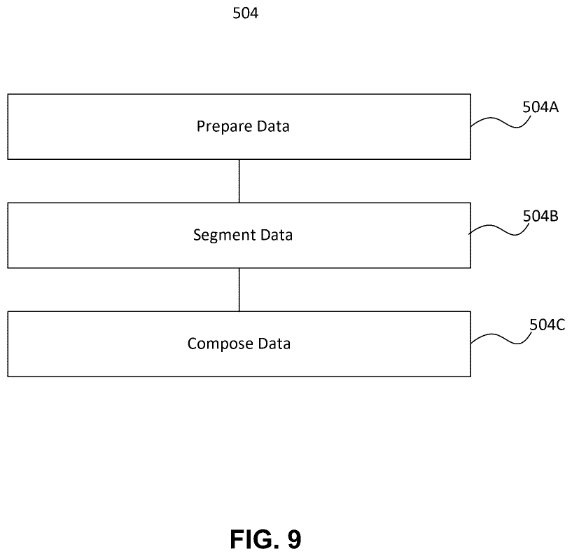

FIG. 9 illustrates a flow diagram for step 504 of process 500, consistent with embodiments of the present disclosure. Step 504 of signal processing may include one or more substeps 504A, 504B, and 504C. In some embodiments, step 504 may be performed by computing system 118. For example, processor 184 may be configured to perform processes described with regards to step 504. Additionally, or alternatively, model(s) 120 may be configured to perform processes described with regard to step 504. It will be appreciated that signal processing can enhance glucose detection, improve blood pressure, vascular health, and metabolic assessments, and significantly enhance non-invasive multi-biomarker monitoring.

Substep 504A may involve preparing data. Data preparation may include the pre-processing of acquired signals (e.g., from step 502). Data processing can reduce noise and enhance signal clarity by filtering out motion artifacts, environmental interference, ambient noise, or the like. In some examples, substep 504A may include time-series analysis, bandpass filtering, baseline correction, and/or Fast Fourier Transforms for noise reduction. For example, to generate PPG waveform that can be interpreted for blood volume changes in microcirculation, substep 504A may involve integrating portions of the selected wavelength ranges. This may include spectral data summation (e.g., aggregating spectral data over wavelength selected ranges, such as 900 to 1000 nm). For example, spectral data summation may involve i.) extracting data points within each selected spectral range (i.e. 900 to 1000 nm); ii.) summing the spectral intensities for each time step to create a composite signal representing the contribution of these wavelengths; iii.) segmenting the summations according to each spectral range (e.g. 610 to 690 nm, 710 to 900 nm, 900 to 970 nm); and iv.) performing additional focalized summations within identified peak and trough segments for specialized signal comparisons. In some embodiments, substep 504A may include signal smoothing. As a non-limiting example, according to

$${S}^{′} ( i ) = \frac{S ( i - 2 ) + S ( i - 1 ) + S ( i ) + S ( i + 1 ) + S ( i + 2 )}{5} ,$$

a 5 point, centered moving average can reduce high frequency noise by i.) computing the average of five consecutive points centered around a middle point; ii.) replacing the center point with the computed average, where the first two and last two data points remain unchanged to preserve edge information. In some embodiments, substep 504A may involve baseline correction. Baseline drift, which can be caused by slow changes in ambient light or physiological movements, can be corrected by applying a linear curve fit over a 5-second window. For example, i.) compute a linear least-squares regression fit for each 5-second segment; ii.) subtract the fitted baseline equation from the raw signal within the window, and iii.) continue sliding the 5-second window to process the entire signal. In some embodiments, substep 504A may include centering and/or signal inversion. For example, after baseline correction, the data can be adjusted to center around zero and inverted to match the expected PPG waveform per wavelength segment, such as by: i.) computing the mean of the corrected signal and subtract it from each data point, or estimate the value that can center the 5 seconds of data around 0; ii.) subtracting the value from each data point and taking the negative of that number; which inverts the signal as would be done in a circuit. (e.g., new signal=−[signal−constant]). In some embodiments, substep 504A may include Fourier series fitting. For example, Fourier series representation with six terms may be fitted to the processed data, where h is the DC offset term, Aₙ are amplitude coefficients, cₙ are phase shifting coefficients, and w is the fundamental frequency, which can provide a mathematical model of the PPG waveform, enabling robust feature extraction, e.g., as a non-limiting example,

$$F ( t ) = h + {A}_{1} sin ( 2 π w t + {c}_{1} ) + {A}_{2} sin ( 4 π w t + {c}_{2} ) + {A}_{3} sin ( 6 π w t + {c}_{3} ) + {A}_{4} sin ( 8 π w t + {c}_{4} ) + {A}_{5} sin ( 10 π w t + {c}_{5} )$$

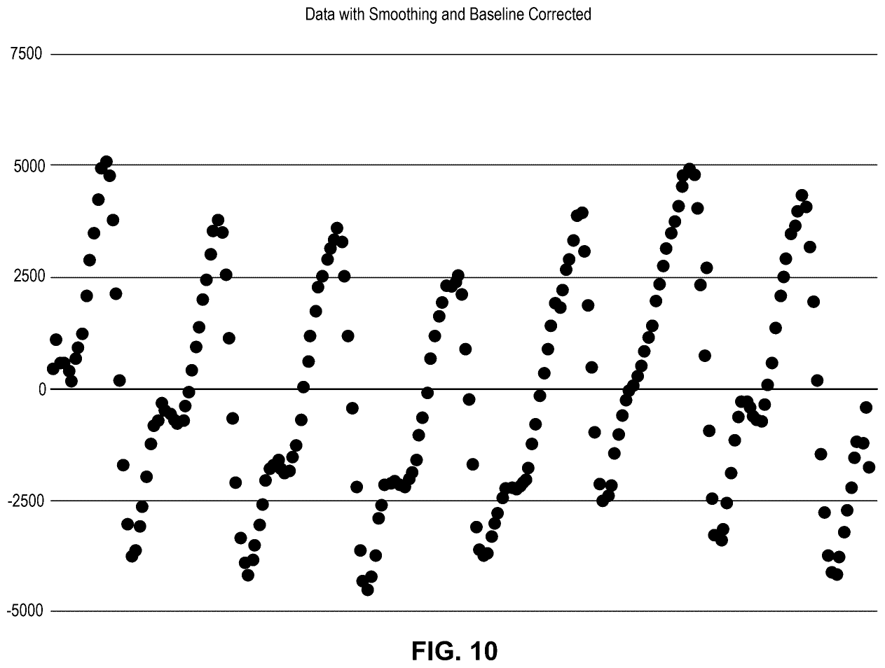

FIG. 10 illustrates an example of processing an acquired signal, consistent with embodiments of the present disclosure. In FIG. 10, it will be appreciated that frequency noise has been reduced in the signal.

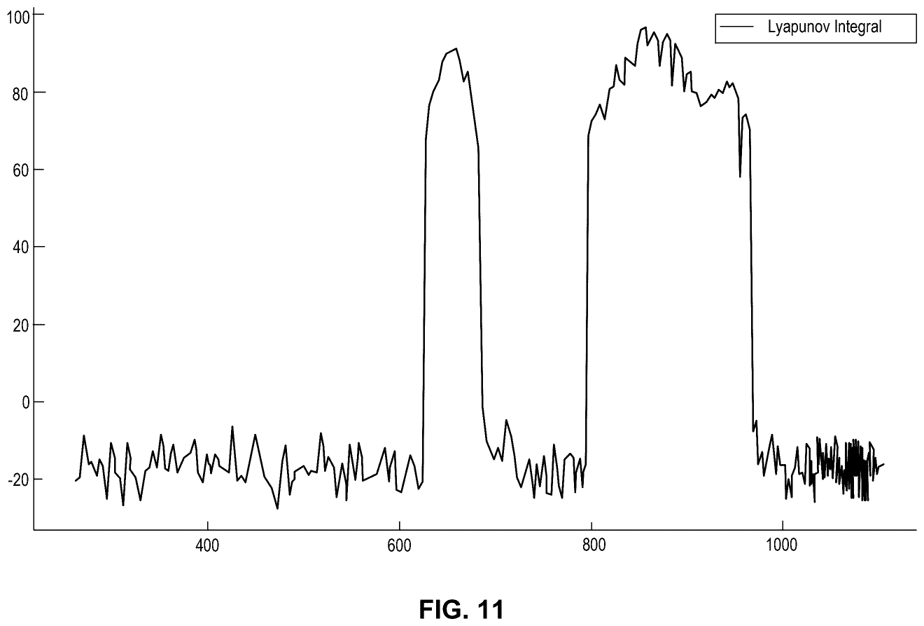

In some embodiments, substep 504A may involve non-linear dynamics and chaos processing to signals. For example, in addition or alternative to processing methods described herein, substep 504A may include Lyapunov Integral Analysis (LIA). Non-linear dynamics, including LIA, may provide data corresponding to system variability. In LIA, a parallel transformation can be implemented to calculate the Lyapunov Exponent and Integral from the raw spectral data, in order to help study the stability and divergence of spectral data over time. FIG. 11 illustrates a graph of LIA applied to a signal, consistent with embodiments of the present disclosure. FIG. 11 illustrates a Lyapunov Integral of a spectral footprint, with 660, 870, and 940 nm selected wavelength ranges. LIA may involve i.) preprocessing data by normalizing intensity values and converting time columns to numerical format; ii.) computing the Lyapunov Exponent by calculating small perturbations in spectral intensity over time and measuring divergence using the largest Lyapunov exponent; and iii.) integrating over time by computing the integral over time to assess spectral system stability. It will be appreciated that LIA may be implemented to measure and analyze the signal stability and variability over time, which when applied to time series data enables improved insights into vascular health, autonomic function, and blood flow regulation.

Referring again to FIG. 9, process 504 may include a substep 504B of segmenting data. In some embodiments, classification and manipulation techniques may be implemented to the PPG data to increase interpretation potential, such as separating each spectral segment PPG signal for feature extraction. Substep 504B may include analyzing formal PPG signal characteristics, mainly periodicity, slopes, phases, and amplitudes, which are attributed to different physiological characteristics. Each segment may be categorized and subsequently compared according to polarizing filter implementation.

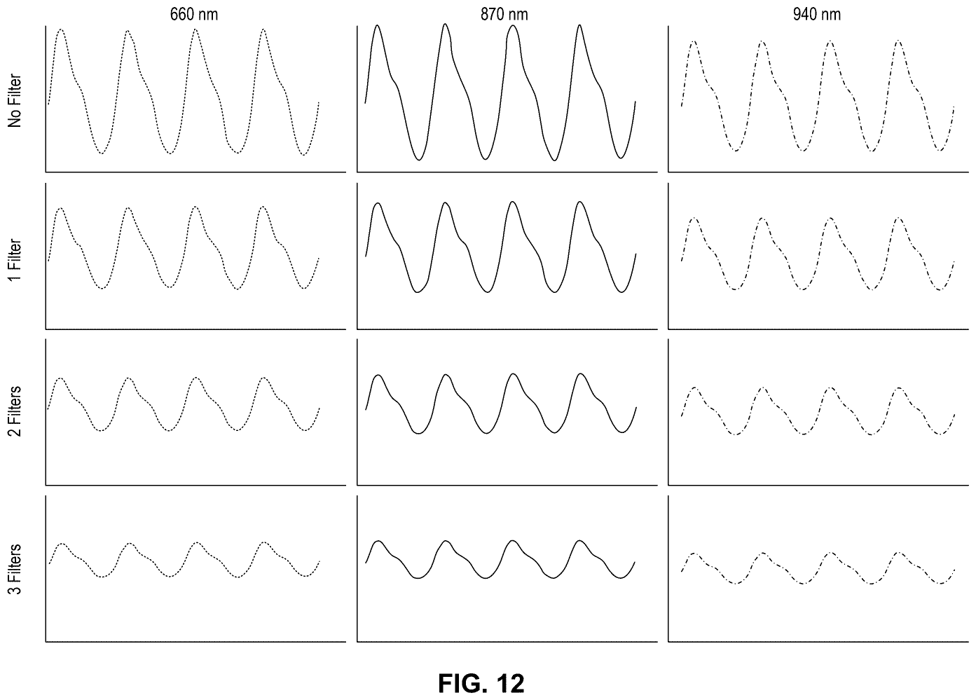

FIG. 12 illustrates an exemplary graph of PPG segmentation, consistent with embodiments of the present disclosure. The graph displays measurements including three selected wavelength ranges (660, 870 and 940 nm) and three sequential linear polarizing filters selected (45°, 0° and 90°), producing 12 unique PPG signals categorized into 3 groups according to spectral range.

Referring again to FIG. 9, process 504 may include a substep 504C of composing data. Composing data may involve generating composite data. Substep 504C may involve labelling and superimposing the PPG signals that have been arranged by spectral segment and polarizing filters (e.g., in substep 504B) into a set of spectral-range dependent PPG composite signals.

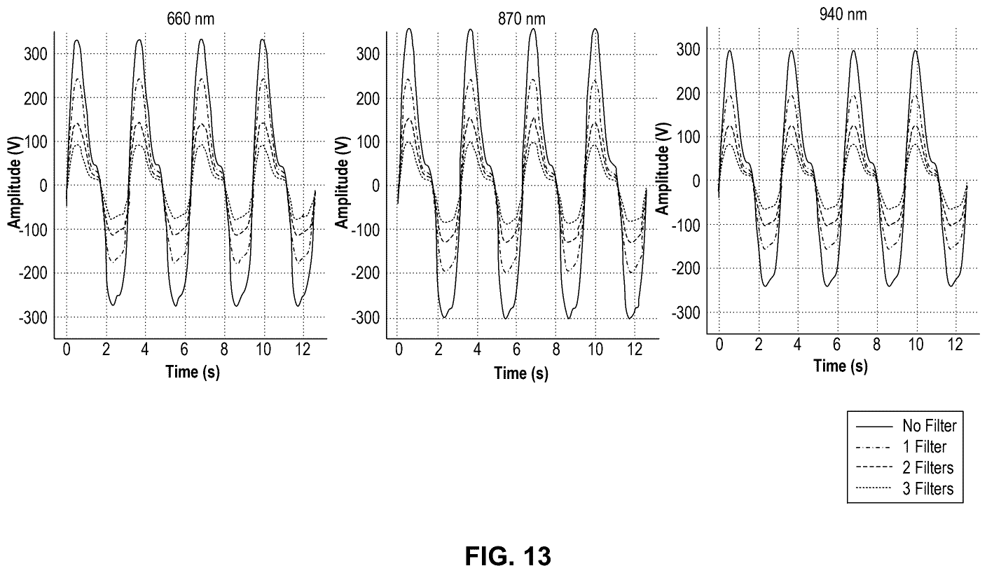

FIG. 13 illustrates an exemplary graph of PPG composition, consistent with embodiments of the present disclosure. The graph displays superimposed PPG signals corresponding to each polarizing filter per spectral segment, which will create a temporal spectral footprint segmented according to wavelength range. It will be appreciated that this will allow for simpler comparisons, feature extraction, and trend analysis.

Referring again to FIG. 5, step 506 may involve extracting features and generating biomarkers. Step 506 may involve extracting features from signals processed in step 504, including from PPG signals, bioimpedance data, and temperature data. For example, extracted features can include 14 PPG features, such as systolic amplitude (e.g., distance between crest and first peak); diastolic amplitude (e.g., distance between crest and second peak); pulse interval (e.g., inter-beat interval or IBI, time between consecutive systolic peaks, representing one cardiac cycle); cardiac period (e.g., pulse interval, duration of one complete cardiac cycle); crest time (e.g., time from the onset of PPG waveform to systolic peak); systolic peak (e.g., time interval of first peak); diastolic peak (e.g., time interval of second peak); dicrotic notch (e.g., time interval of local minimum between systolic and diastolic peaks); pulse width (e.g., duration between the onset and the end of the PPG pulse at a specified amplitude); systolic/diastolic interval (e.g., time between the systolic peak and the diastolic peak or dicrotic notch); systolic phase (e.g., duration from the onset of the PPG waveform to the systolic peak); diastolic phase (e.g., duration from the systolic peak to the end of the PPG waveform); Velocity Plethysmogram (VPG, e.g., first derivative of the PPG related to the rate of blood volume change in the arteries); and Acceleration Plethysmogram (APG, e.g., second derivative of the PPG related to the acceleration of deceleration of blood flow).

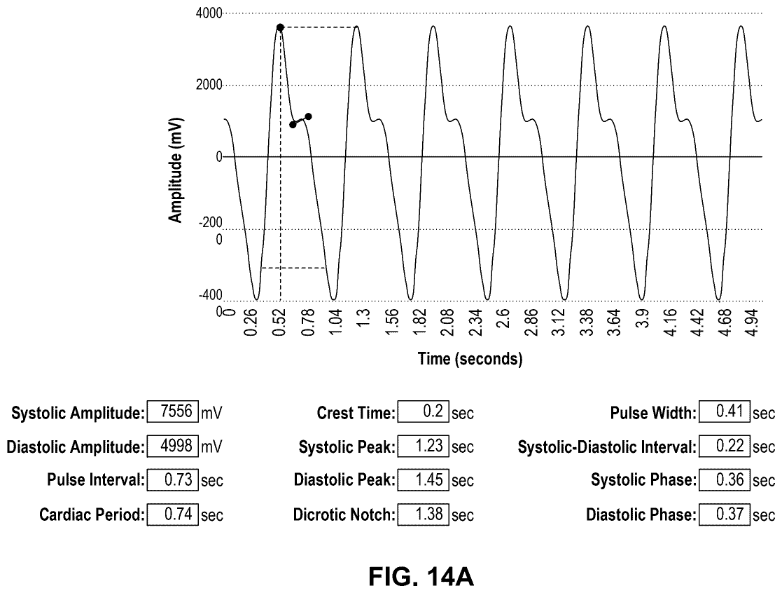

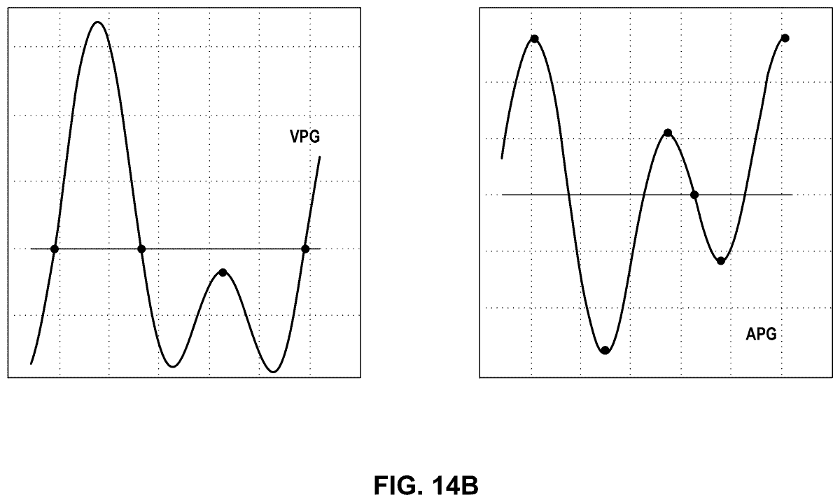

FIGS. 14A-14B illustrate extracted features, consistent with embodiments of the present disclosure. FIG. 14A displays systolic amplitude, diastolic amplitude, pulse interval, cardiac period, crest time, systolic peak, diastolic peak, dicrotic notch, pulse width, systolic-diastolic interval, systolic phase, and diastolic phase. FIG. 14B illustrates VPG and APG. As a non-limiting example, VPG (F′(t) and APG (F″(t), respectively) may be calculated as

$${F}^{′} ( t ) = 2 π {A}_{1} w cos ( {c}_{1} + 2 π w t ) + 4 π {A}_{2} w cos ( {c}_{2} + 4 π w t ) + 6 π {A}_{3} w cos ( {c}_{3} + 6 π wt ) + 8 π {A}_{4} w cos ( {c}_{4} + 8 π w t ) + 10 π {A}_{5} w cos ( {c}_{5} + 10 π w t ) + C \\ {F}^{″} ( t ) = - 4 {π}^{2} {A}_{1} {w}^{2} sin ( {c}_{1} + 2 π w t ) - 16 {π}^{2} {A}_{2} {w}^{2} sin ( {c}_{2} + 4 π wt ) - 36 {π}^{2} {A}_{3} {w}^{2} sin ( {c}_{3} + 6 π w t ) - 64 {π}^{2} {A}_{4} {w}^{2} sin ( {c}_{4} + 8 π wt ) - 100 {π}^{2} {A}_{5} {w}^{2} sin ( {c}_{5} + 10 π w t )$$

In some embodiments, step 506 may involve generating Biomarker Specific Features (BSFs) from the extracted features. BSFs may be used to estimate biomarkers, and can enable enhanced interpretation of signals and improved prediction of biomarkers. In some embodiments, Biomarker Specific Features can include heart rate, heart rate variability, oxygen saturation, systolic blood pressure, diastolic blood pressure, vascular age, arterial compliance, perfusion index, respiratory rate, and blood glucose. Heart rate may focus on peaks (e.g., pulse Interval (PI) or Normal Beats (NN)). The periodic nature of the PPG signal (which corresponds to cardiac cycles). HR may be calculated by dividing 60 (seconds) by the interval between successive peaks (Pulse Interval (P)), e.g., as a non-limiting example, HR (BPM)=60/Pulse Interval (seconds).

Heart rate variability (HRV) may focus on successive peaks (Pulse Interval (PI) or Normal Beats (NN)). HRV may be calculated by variations in time intervals between consecutive heartbeats, such as by observing the standard deviation of NN (Normal Beat) intervals (SDNN). For example, HRV may involve applying a Discrete Fourier Transform (DFT) to transform and filter NN interval sequence; fitting a Gaussian Regression to the Power Spectral Density (PSD) of the transformed NN intervals; and calculating the Standard Deviation of the fitted Gaussian function in the HRV spectrum (σHRV) times 2/Pi (to normalize HRV comparisons): e.g., as a non-limiting example, HRV=σHRV×2/π).

Oxygen saturation (e.g., SpO₂) may focus on red/infrared absorbance ratio. For example, ratio of the amplitudes of the PPG signals at 660 nm (red) and the infrared range (870 and 940 nm),

$${R}_{local} = \frac{( \frac{Max Red - Min Red}{Min Red} )}{\frac{Max IR - Min IR}{Min IR}}$$

as a non-limiting example, where maximum signal is the pulsatile component, minimum signal is the non-pulsatile (baseline) component.

Systolic blood pressure (SBP) may focus on Slopes, Pulse Wave Velocity, and Pulse Transit Time. Determining systolic blood pressure may involve an estimation of the Pulse Wave Velocity (PWV) based on PPG slope and systolic and diastolic period durations, which yields the time difference between systolic and diastolic peaks. SBP may be influenced by the steepness of the systolic upstroke, the PPG slope during systole and the pulse transit time (PTT) are strong indicators. In a non-limiting example, SBP=Aₛ·PPG Amplitude+Bₛ·PPG Width+Cₛ·PPG Slope (Systolic)+Dₛ, where PPG Amplitude=height from the diastolic trough to the systolic peak, PPG Width=time between the foot (start) of the PPG wave and the dicrotic notch (end of systolic phase). PPG Slope (Systolic)=rate of rise from the PPG foot to the systolic peak. As−Dₛ=calibration constants.

Diastolic blood pressure (DBP) may focus on slopes, Pulse Wave Velocity, Pulse Transit Time. Determining DBP may involve an estimation of the Pulse Wave Velocity (PWV) based on PPG slope and systolic and diastolic period durations, which yields the time difference between systolic and diastolic peaks. DBP may be primarily influenced by vascular resistance, wave reflection, and the falling phase of the PPG waveform. In a non-limiting example, DBP=A_{d}·PPG Amplitude+B_{d}·PPG Width+C_{d}·PPG Slope (Diastolic)+D_d, where PPG Amplitude=Peak-to-trough height (difference between systolic and diastolic peaks, PPG Width=Total duration of the PPG cycle (influences pulse wave velocity estimation). PPG Slope (Diastolic)=Rate of fall from systolic peak to diastolic trough. Ad−D_{d}=Calibration constants.

Vascular age may focus on analysis of APG and vascular ageing index. Determining vascular age may involve a calculation of the Vascular Ageing Index (VAI) based on the ratios of APG wave amplitudes: In a non-limiting example, VAI=|b|+|c|+|d|+|e|/|a|, and an estimation of arterial health (Vascular Age) compared to normative age-related data through non-linear regression or empirical equations, such as, in a non-limiting example, VA=A·log(VAI)+B, Where A, B are calibration constants, and log(VAI) ensures a smooth age progression (since vascular aging is nonlinear).

Arterial compliance may focus on analysis of APG and Arterial Stiffness Index. Determining arterial compliance may involve the subject's height divided by the time between the systolic peak and the reflected wave (inflection point): ASI=Height/ΔT. Deriving Arterial Compliance from arterial stiffness index may involve PWV and hemodynamic relationships, such as

$$AC \approx \frac{1}{ρ} \cdot k 2 {ASI}^{2} ,$$

in a non-limiting example, where: ρ=1.06 g/cm3 (blood density), k is an empirical calibration factor.

Perfusion index (PI) may focus on pulsatile AC/non-pulsatile DC ratio. Determining perfusion index may involve comparing the pulsatile (AC) component of the signal to the non-pulsatile (DC) component, e.g., perfusion index=(AC Component/DC Component)×100. Perfusion index may also include a weighted transformation to stabilize fluctuations in low-perfusion conditions, such as in a non-limiting example, PI_{enhanced}=log(1+AC Component/DC Component).

Respiratory rate may focus on low frequency oscillations. Determining respiratory rate may involve calculating changes in PPG amplitude (due to changes in blood volume with respiration), changes in pulse rate variability (PRV) (due to respiratory sinus arrhythmia (RSA)), and slow baseline shifts (due to respiratory-induced thoracic pressure changes). Determining respiratory rate may involve applying a bandpass filter to isolate respiratory components e.g., in a non-limiting example, respiratory rate=Peak Frequency (Hz)×60.

Blood glucose may focus on HRV correlation and AC residual absorbance comparisons. Determining blood glucose may involve an indirect estimation by analyzing changes in the PPG signal's amplitude and frequency (HRV) at a relevant infrared spectral range (i.e. HRV at 940 nm (and all respective polarizing filters)), which correlates with glucose affecting osmotic pressure and blood volume in the measurement window HRV (940 nm F0, F1, F2, F3)=σHRV×2/π. Determining blood glucose may involve observing optical rotation changes due to glucose concentration differences by contrasting filter-specific absorbance between different wavelength ranges (AC/DC ratios), such as in a non-limiting example,

$${R}_{C} = {AC}_{600} \cdot \frac{{DC}_{800}}{{AC}_{800}} \cdot {DC}_{600} \\ {R}_{G 1} = {AC}_{800} \cdot \frac{{DC}_{900}}{{AC}_{900}} \cdot {DC}_{800} \\ {R}_{G 2} = {AC}_{600} \cdot \frac{{DC}_{900}}{{AC}_{900}} \cdot DC\_ 600$$

where R_{c} is the AC/DC ratio of red (i.e. 660 nm) and NIR₁ (i.e. 870 nm), R_{G1} is the AC/DC ratio of NIR₁ (i.e. 870 nm) and NIR₂ (i.e. 940 nm), and R_{G2} is the AC/DC ratio of red (i.e. 660 nm) and NIR₂ (i.e. 940 nm). Determining blood glucose may involve calculating the ratio between wavelength range ratios according to polarizing filters, such as in a non-limiting example,

$${R}_{c}^{LR} = {R}_{c}^{F 0} \cdot {R}_{c}^{F 1} \cdot {R}_{c}^{F 2} \cdot {R}_{c}^{F 3} ) \\ {R}_{G 1}^{LR} = {R}_{G 1}^{F 0} \cdot {R}_{G 1}^{( F 1 )} \cdot {R}_{G 1}^{F 2} \cdot {R}_{G 1}^{F 3} \\ {R}_{G 2}^{LR} = {R}_{G 2}^{F 0} \cdot {R}_{G 2}^{( F 1 )} \cdot {R}_{G 2}^{F 2} \cdot {R}_{G 2}^{F 3}$$

where R_{c}^{(LR)} is the ratio of R_{c} per applied polarizing filter (i.e. F0 means no filters, F1 means one filter, F2 means 2 filters, and F3 means 3 filters). R_{G1}^{(LR)} and R_{G2}^{(LR)} have the same conditions.

Referring to FIG. 5, process 500 may include a step 508 of analyzing biomarkers, features, and/or signals. Step 508 may involve analyzing Biomarker Specific Features (e.g., generated in step 506) as well as other data obtained from device 101 and/or computing system 118, such as extracted features and/or acquired signals. Analyzing biomarkers can involve analyzing subject data (e.g., demographic data, anthropometric data, bioimpedance data, temperature data, environmental data, health history data), as well as measurement parameter data (e.g., scans, wavelength ranges, light pattern, integration time, polarizing filters & pattern, measurement mode, dark measurement, light path, device calibration); signals corresponding to spectral footprint data, such as segmented and composed PPG data, extracted features, and Biomarker Specific Features, and processed signal data (e.g., Lyapunov Integral), as described herein. In some embodiments, step 508 may involve detecting and/or filtering noisy subsegments of data to select stable, high quality data. Data may be assessed based on their dominant frequency stability and other statistical properties. The criteria for selecting segments may be influenced by the biomarkers and biophysiological variables that will be predicted. For example, for HR estimation, segments with a stable and temporally consistent dominant frequency may be identified, for SpO₂ predictions, additional criteria may be applied to ensure both frequency stability and amplitude consistency. In some embodiments, step 508 may involve applying machine learning models to the aforementioned data points, as well as additional data points, such complementary data. For example, model(s) 120 may include preparatory machine learning models that can be applied to the data. Preparatory models can provide feature categorization, further feature extraction, interpretation, evaluation, and refinement, such as univariate models. Preparatory models can provide initial estimates of Biomarkers and Biomarker Specific Features. The preparatory models may be trained to evaluate the quality of extracted features by computing univariate correlations with ground truth biomarker measurements. For example, correlation metrics can include Pearson's r, Spearman's ρ, and Kendall's τ. In some examples, based on the correlation, certain subsegments of data identified as having lower noise can be used for further analysis. In some embodiments, machine learning models of model(s) 120 can be trained on data described herein, as well as additional training data, such as subject vitals (e.g., body temperature, HR, blood pressure), waist circumference, hip circumference, or the like, as well as body composition data, such as total body water, body fat mass, BMI, or the like, as well as blood chemistry, such as serum glucose, Hemoglobin A1c (HbA1c), protein, cholesterol levels, or the like. For example, storage 182 can include training data. In some examples, step 508 may involve organizing measurements into a vectorized and/or dimensionally-reduced format (e.g., by Principal Component Analysis).

In some embodiments, step 508 may involve applying classical and deep learning machine learning models. For example, model(s) 120 may include classical and deep learning machine learning models. Classical models may include sparse parametric, non-parametric, and partition-based models, as non-limiting examples. For example, classical models can include regression models (e.g., linear, logistic, lasso, random forest regression, gaussian process, support vector, XGBoost regression), support vector machines, Hidden Markov Models, Naïve Bayes models, or the like. Deep learning models may assist in optimizing trend-based estimations. Deep learning models may be able to adapt to the complexity of data. For example, Deep learning models can include K-Nearest Neighbors, decision trees, random forests, neural networks (e.g., Recurrent Neural Networks, Convolutional Neural Networks), transformers, multimodal or multitask models, or the like. Deep learning models may assist in making homologous biomarker predictions from different and complex identified correlation, such as making cross-biomarker predictions. For example, in step 508, preparatory models may generate initial biomarker predictions, and classical models and deep learning models can each analyze the biomarker data to supplement such initial biomarker predictions.

In some embodiments, process 500 may include a step 510 of generating an ensemble biomarker determination. Step 510 may involve ensemble modeling (e.g., a jury system) to optimize biomarker predictions from both classical and deep learning model analysis (e.g., from step 508). The ensemble biomarker determination may be a weighted comparison of model outputs or predictions for each biomarker. Step 510 may involve adjusting model weighting based on biomarker type, physiological correlations, and data availability. Step 510 may involve multi-model voting mechanisms, which can provide robust estimations and reduce bias. For example, a biomarker ensemble determination may involve a weighted estimation (e.g., of predictions from classical models, deep learning models, and preparatory models) applied to each Biomarker Specific Feature. Step 510 may expand the extracted features with derived features capturing distribution properties, providing additional insights (e.g., for new patient cohorts and individuals). Input and label transformations may be programmed to facilitate transfer learning and domain adaptation for generalization to new patient cohorts and individuals. Density-based techniques may also be applied to sample weighting according to label distributions. In some examples, different feature scaling methods and group penalties are used to optimize performance for groups of inputs. In some embodiments, step 510 may involve generating an Overall Health Index from the weighted ensemble biomarker determination. The weighted ensemble biomarker determination may be used to provide an Overall Health Index, which can include scores corresponding to healthcare status based on the biomarker predictions. In some embodiments, step 510 may involve providing the Overall Health Index to a user interface of interface(s) 160. For example, step 510 may include generating a user interface displaying the Overall Health Index, health predictions, recommendations (e.g., and clinical feedback mechanisms), as well as alerting systems for detection and/or notification of health status decline or changes.

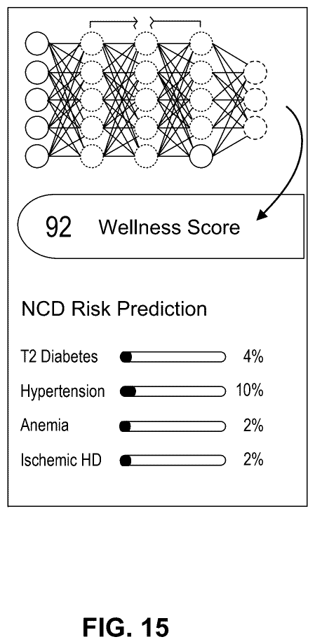

FIG. 15 a depiction of an Overall Health Index, consistent with embodiments of the present disclosure. For example, FIG. 15 may be an illustration of a user interface of interface(s) 160 displayed on communication device 116. The Overall Health Index (e.g., generated in step 510) may be a score or metric representing wellness or health status for a subject or group of subjects. For example, the Overall Health Index may include predictions of disease or condition risk, including non-communicable conditions such as Diabetes, Hypertension, Anemia, or Ischemic Heart Disease. The Overall Health Index may be generated based on relationships between various data analyzed in process 500, including Biomarker Specific Features, acquired signals, complementary data, or the like.

In some embodiments, process 500 and applications of device 101 may involve calibration. For example, substep 502C may involve calibration of device 101. Calibration may involve generating calibration curves or coefficients specific to device 101. Calibration may enable dynamically adjusting for temperature fluctuations, environmental variations (altitude, climate conditions), biomarker concentration ranges, and other physiological and environmental factors to provide standardized performance validation and measurement accuracy. In some embodiments, a benchtop calibration process that includes device-specific calibration curves and coefficients for spectral signal standardization, as well as biomarker-specific spectral signals of various known concentrations and levels to provide a dynamic range of biomarker signals for continuous training. For example, to optimize signal reproducibility and standardization, an optical calibration chamber may be used to test device 101 under different conditions that may affect spectral behavior. An optical chamber may test positioning and angle differences of each available light source for sensor hypersensitivity or hyposensitivity, temperature and humidity effects across different integration times and lighting conditions, electronic noise conditions of all different boards, polarizing filter efficiency, and verify sensor calibration coefficients, which can be adjusted if necessary, to provide device-specific calibration curves. Additionally, or alternatively, for biomarker-specific spectral dynamic ranges, a Hand Phantom that can mimic human spectral signals may be used. For example, calibration with a Hand Phantom may allow for acquisition of optical signals of heart rate, heart rate variability, oxygen saturation, systolic blood pressure, diastolic blood pressure, and blood glucose. These signals may be identified by re-creating the expected spectral conditions for each biomarker in human-like fingers with microvascular controllable circulation and tissue-like turbid materials that can dilute concentrations of different molecules. Such measurements may be made by device 101 after correlation to ground truth biomarker measurements. Following benchtop calibration, device 101 may run a calibration procedure to verify that light sources and sensors are operating within standards. For example, calibration may involve a stable spectral signal that achieves specific intensity readings with preset integration times, and adjusting light source drive current and positioning if necessary. In some embodiments, calibration may involve an additional step for each individual measurement. This includes identifying the individual to be measured by User ID and adjusting the tunable measurement parameters to their specific characteristics. A short spectral test can also be performed to ensure correct sample positioning and prevent sensor saturation without polarizing filters, while preserving all spectral information with all selected polarizing filters.

Embodiments of the present disclosure may be described with respect to the following clauses:
1. A portable electronic device for biosignal acquisition comprising:
   a housing having a chamber configured to receive a sample;
   a light source array disposed adjacent to the chamber, wherein the light source array is configured to emit light for transmission through the sample;
   a plurality of sensors configured to detect a plurality of signals from the sample, the plurality of sensors comprising a bioimpedance sensor, a spectrometer sensor, and an infrared temperature sensor;
   a tunable filter array comprising a plurality of polarizing filters, wherein one or more polarizing filters of the plurality of polarizing filters are arranged in parallel to each other, oriented perpendicularly to the emitted light, and disposed between the light source array and the spectrometer sensor; and
   a communications module configured to transmit the plurality of signals;
   wherein the light source array, the plurality of sensors, the tunable filter array, and the communications module are each disposed within the housing.
2. The device of clause 1, wherein the light source array is configured to emit light at a plurality of wavelengths and pulsating frequencies.
3. The device of any of clauses 1-2, wherein the plurality of polarizing filters comprises at least two polarizing filters each having a different polarization state.
4. The device of any of the preceding clauses, wherein the chamber is configured to receive the sample between the light source array and the spectrometer sensor, wherein one or more polarizing filters of the plurality of polarizing filters are disposed between the light source array and where the chamber is configured to receive the sample.
5. The device of any of the preceding clauses, wherein the one or more polarizing filters of the plurality of polarizing filters are arranged in parallel to each other and disposed between where the chamber is configured to receive the sample and at least one sensor of the plurality of sensors.
6. The device of any of the preceding clauses, wherein the sample is a peripheral anatomical sample comprising a vascular anatomical segment.
7. The device of any of the preceding clauses, wherein the communications module is configured to transmit the plurality of signals to a computing system for generating a weighted ensemble biomarker determination.
8. A method performed by at least one processor, the method comprising:
   receiving physiological signals for a subject from a device, the physiological signals including temperature, bioimpedance, and light absorbance measurements;
   generating a spectral footprint signal from the received physiological signals, the spectral footprint signal including Photoplethysmography (PPG) and system variability data;
   processing the spectral footprint signal, wherein the processing includes segmenting the PPG data and generating superimposed PPG composite data;
   extracting features from the composed PPG data to generate biomarker specific features;
   analyzing, with one or more machine learning models, the biomarker specific features; and
   generating, based on the analysis, a weighted ensemble biomarker determination.
9. The method of clause 8, further comprising analyzing, with the one or more machine learning models, the biomarker specific features, the physiological signals, the superimposed PPG data, and complementary data, wherein the segmenting and generating superimposed composite data are based on a plurality of wavelengths of the PPG data.
10. The method of any of clauses 8-9, wherein the biomarker specific features include at least one of heart rate, heart rate variability (HRV), oxygen saturation, systolic blood pressure, diastolic blood pressure, vascular age estimation, arterial compliance, perfusion index, and respiration rate, and blood glucose.
11. The method of clauses 8-10, wherein the one or more machine learning models comprise classical machine learning models and deep learning models, and wherein applying the one or more machine learning models comprises applying both the classical machine learning models and the deep learning model to at least one of the biomarker specific features, physiological signals, segmented PPG signals and superimposed composite PPG signals, and complementary data.
12. A system for biomarker analysis, the system comprising:
   a device comprising:
      a chamber configured to receive a sample;
      a light source array disposed adjacent to the chamber, wherein the light source array is configured to emit light for transmission through the sample;
      a plurality of sensors configured to detect a plurality of signals from the sample, the plurality of sensors comprising a bioimpedance sensor, a spectrometer sensor, and an infrared temperature sensor; and
      a communications module configured to transmit the plurality of signals; and
a computing system in electronic communication with the device, the computing system comprising:
   one or more machine learning models;
   one or more memory devices storing executable instructions; and
   at least one processor configured to execute instructions to perform operations comprising;
      receiving the plurality of signals from the device;
      generating biomarker specific features from the plurality of signals; and
      applying the one or more machine learning models to the biomarker specific features to generate a weighted ensemble biomarker determination.
13. The biomarker analysis system of clause 12, wherein the operations further comprise:
   generating a spectral footprint signal from the received signals, the spectral footprint signal including Photoplethysmography (PPG) data and system variability data.
14. The biomarker analysis system of any of clauses 12-13, wherein the operations further comprise:
   processing the spectral footprint signal, wherein the processing includes segmenting the PPG data and generating superimposed PPG composite data; and
   extracting features from the composed PPG data to generate the biomarker specific features.
15. The biomarker analysis system of any of clauses 12-14, wherein the one or more machine learning models of the computing system comprise classical machine learning models and deep learning models, and wherein applying the one or more machine learning models comprises applying both the classical machine learning models and the deep learning model to at least one of the biomarker specific features, the plurality of signals, the segmented PPG signals and superimposed composite PPG signals, and complementary data.
16. The biomarker analysis system of any of clauses 12-15, wherein the device further comprises a tunable filter array having a plurality of polarizing filters.
17. The biomarker analysis system of any of clause 12-16, wherein the plurality of polarizing filters comprises at least two polarizing filters each having a different polarization state.
18. The biomarker analysis system of any of clauses 12-17, wherein the sample includes a vascular anatomical segment.
19. The biomarker analysis system of any of clauses 12-18, wherein the biomarker specific features include at least one of heart rate, heart rate variability (HRV), oxygen saturation, systolic blood pressure, diastolic blood pressure, vascular age estimation, arterial compliance, perfusion index, and respiration rate, and blood glucose.
20. The biomarker analysis system of any of clauses 12-19, wherein the chamber, plurality of sensors, light source array, and communications module are each disposed within a housing of the device.

It will be apparent to those skilled in the art that various modifications and variations can be made to the environment instantiation platform. While illustrative embodiments have been described herein, the scope of the invention includes any and all embodiments having equivalent elements, modifications, omissions, combinations (e.g., of aspects across various embodiments), adaptations and/or alterations as would be appreciated by those skilled in the art based on the present disclosure. It is also intended that the sequence of steps shown in figures are only for illustrative purposes and are not intended to be limited to any particular sequence of steps. As such, those skilled in the art can appreciate that these steps can be performed in a different order, or performed with steps omitted, while implementing the same method. The limitations in the claims are to be interpreted broadly based on the language employed in the claims and not limited to examples described in the present specification or during the prosecution of the application, which examples are to be construed as non-exclusive. It is intended, therefore, that the specification and examples be considered as exemplary only, with a true scope and spirit of the invention being indicated by the following claims and their full scope of equivalents.

What is claimed is:

1. A portable electronic device for biosignal acquisition comprising:
a housing having a chamber configured to receive a sample;
a light source array disposed adjacent to the chamber, wherein the light source array is configured to emit light for transmission through the sample;
a plurality of sensors configured to detect a plurality of signals from the sample, the plurality of sensors comprising a bioimpedance sensor, a spectrometer sensor, and an infrared temperature sensor;
a tunable filter array comprising a plurality of polarizing filters, wherein one or more polarizing filters of the plurality of polarizing filters are oriented perpendicularly to the emitted light and disposed between the light source array and the spectrometer sensor; and
a communications module configured to transmit the plurality of signals;
wherein the light source array, the plurality of sensors, the tunable filter array, and the communications module are each disposed within the housing.

2. The device of claim 1, wherein the light source array is configured to emit light at a plurality of wavelengths and pulsating frequencies.

3. The device of claim 1, wherein the plurality of polarizing filters comprises at least two polarizing filters each having a different polarization state.

4. The device of claim 1, wherein the chamber is configured to receive the sample between the light source array and the spectrometer sensor, wherein the one or more polarizing filters of the plurality of polarizing filters are arranged in parallel to each other and disposed between the light source array and where the chamber is configured to receive the sample.

5. The device of claim 1, wherein the one or more polarizing filters of the plurality of polarizing filters are arranged in parallel to each other and disposed between where the chamber is configured to receive the sample and at least one sensor of the plurality of sensors.

6. The device of claim 1, wherein the sample is a peripheral anatomical sample comprising a vascular anatomical segment.

7. The device of claim 1, wherein the communications module is configured to transmit the plurality of signals to a computing system for generating a weighted ensemble biomarker determination.

8. A method performed by at least one processor, the method comprising:
receiving physiological signals for a subject from a device, the physiological signals including temperature, bioimpedance, and light absorbance measurements;
generating a spectral footprint signal from the received physiological signals, the spectral footprint signal including Photoplethysmography (PPG) and system variability data;
processing the spectral footprint signal, wherein the processing includes segmenting the PPG data and generating superimposed PPG composite data;
extracting features from the composed PPG data to generate biomarker specific features;
analyzing, with one or more machine learning models, the biomarker specific features; and
generating, based on the analysis, a weighted ensemble biomarker determination.

9. The method of claim 8, further comprising analyzing, with the one or more machine learning models, the biomarker specific features, the physiological signals, the superimposed PPG data, and complementary data, wherein the segmenting and generating superimposed composite data are based on a plurality of wavelengths of the PPG data.

10. The method of claim 8, wherein the biomarker specific features include at least one of heart rate, heart rate variability (HRV), oxygen saturation, systolic blood pressure, diastolic blood pressure, vascular age estimation, arterial compliance, perfusion index, and respiration rate, and blood glucose.

11. The method of claim 8, wherein the one or more machine learning models comprise classical machine learning models and deep learning models, and wherein applying the one or more machine learning models comprises applying both the classical machine learning models and the deep learning model to at least one of the biomarker specific features, physiological signals, segmented PPG signals and superimposed composite PPG signals, and complementary data.

12. A system for biomarker analysis, the system comprising:
a device comprising:
a chamber configured to receive a sample;
a light source array disposed adjacent to the chamber, wherein the light source array is configured to emit light for transmission through the sample;
a plurality of sensors configured to detect a plurality of signals from the sample, the plurality of sensors comprising a bioimpedance sensor, a spectrometer sensor, and an infrared temperature sensor; and
a communications module configured to transmit the plurality of signals; and
a computing system in electronic communication with the device, the computing system comprising:
one or more machine learning models;
one or more memory devices storing executable instructions; and
at least one processor configured to execute instructions to perform operations comprising;
receiving the plurality of signals from the device;
generating biomarker specific features from the plurality of signals; and
applying the one or more machine learning models to the biomarker specific features to generate a weighted ensemble biomarker determination.

13. The biomarker analysis system of claim 12, wherein the operations further comprise:
generating a spectral footprint signal from the received signals, the spectral footprint signal including Photoplethysmography (PPG) data and system variability data.

14. The biomarker analysis system of claim 13, wherein the operations further comprise:
processing the spectral footprint signal, wherein the processing includes segmenting the PPG data and generating superimposed PPG composite data; and
extracting features from the composed PPG data to generate the biomarker specific features.

15. The biomarker analysis system of claim 14, wherein the one or more machine learning models of the computing system comprise classical machine learning models and deep learning models, and wherein applying the one or more machine learning models comprises applying both the classical machine learning models and the deep learning model to at least one of the biomarker specific features, the plurality of signals, the segmented PPG signals and superimposed composite PPG signals, and complementary data.

16. The biomarker analysis system of claim 12, wherein the device further comprises a tunable filter array having a plurality of polarizing filters.

17. The biomarker analysis system of claim 16, wherein the plurality of polarizing filters comprises at least two polarizing filters each having a different polarization state.

18. The biomarker analysis system of claim 12, wherein the sample includes a vascular anatomical segment.

19. The biomarker analysis system of claim 12, wherein the biomarker specific features include at least one of heart rate, heart rate variability (HRV), oxygen saturation, systolic blood pressure, diastolic blood pressure, vascular age estimation, arterial compliance, perfusion index, and respiration rate, and blood glucose.

20. The biomarker analysis system of claim 12, wherein the chamber, plurality of sensors, light source array, and communications module are each disposed within a housing of the device.
